(19) United States
(12) Patent Application Publication
(10) Pub. No.: US 2026/0157939 A1
(43) Pub. Date: Jun. 11, 2026

(54) BIOACTIVE GLASS COMPOSITIONS AND METHODS FOR PERIODONTAL THERAPIES AND TOOTH STABILIZATION

(71) Applicant: ReDyne, LLC, San Juan, PR (US)

(72) Inventors: Jeff FRANCO, San Juan, PR (US); Kemal SCHANKERELI, Stillwater, MN (US); Richard MURPHY, White Bear TWP, MN (US)

(21) Appl. No.: 19/411,091

(22) Filed: Dec. 5, 2025

Related U.S. Application Data
(60) Provisional application No. 63/729,156, filed on Dec. 6, 2024.

(51) Int. Cl. A61K 6/77; A61C 19/06; A61K 9/00; A61K 33/14; A61K 33/22; A61K 47/02

(52) U.S. Cl. CPC A61K 6/77; A61C 19/063; A61K 9/0063; A61K 33/14; A61K 33/22; A61K 47/02

(57) ABSTRACT

Provided herein are bioactive glass delivery system for periodontal therapy including a cannula having a proximal end and a distal end with an internal lumen extending therebetween, bioactive glass fiber material loaded within the internal lumen wherein the bioactive glass fiber material may include boron oxide and calcium oxide as primary network formers and one or more trace elements selected from copper oxide, zinc oxide, strontium oxide, iron oxide, potassium oxide, magnesium oxide, sodium oxide, and phosphorus pentoxide, and an obturator positioned within the internal lumen and configured to advance the bioactive glass fiber material through the distal end of the cannula.

### CROSS-REFERENCE TO RELATED APPLICATIONS

This application claims priority to U.S. Provisional Application No. 63/729,156, filed on Dec. 6, 2024, which is hereby incorporated by reference in its entirety.

### FIELD OF INVENTION

The present disclosure relates to dental and periodontal therapies, and more particularly to boron-based bioactive glass compositions and methods for treating periodontal diseases, promoting bone regeneration, stabilizing loose teeth, and enhancing dental implant performance.

### BACKGROUND

Periodontal disease represents a widespread condition affecting the supporting structures of teeth, including the gums, periodontal ligament, and alveolar bone. This progressive condition may lead to increased tooth mobility, gum recession, and eventual tooth loss when left untreated. The disease typically results from a combination of bacterial infection, host immune responses, and environmental factors, with bacterial pathogens such as Porphyromonas gingivalis, Treponema denticola, Tannerella forsythia, and Fusobacterium nucleatum playing prominent roles in disease progression.

Conventional treatment approaches, including scaling and root planing, focus on reducing calculus and bacterial loads to restore periodontal health. However, these methods may prove insufficient for addressing advanced periodontal disease, particularly in cases involving residual bone loss. Traditional cleaning procedures typically do not provide mechanisms for stabilizing already loose teeth or promoting active bone regeneration in affected areas.

There remains a need for improved materials and delivery systems that can address both the biological and mechanical challenges associated with periodontal disease treatment and dental implant integration. Such systems may benefit from materials that provide antimicrobial properties, promote bone regeneration, and offer versatile delivery mechanisms suitable for various clinical scenarios.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a bioactive glass delivery system for periodontal therapy is provided. The bioactive glass delivery system includes a cannula having a proximal end and a distal end with an internal lumen extending therebetween. The system includes bioactive glass material loaded within the internal lumen, wherein the bioactive glass fiber material comprises boron oxide and calcium oxide as primary network formers and one or more trace elements selected from copper oxide, zinc oxide, strontium oxide, iron oxide, potassium oxide, magnesium oxide, sodium oxide, and phosphorus pentoxide. The system includes an obturator positioned within the internal lumen and configured to advance the bioactive glass fiber material through the distal end of the cannula.

According to other aspects of the present disclosure, the bioactive glass delivery system may include one or more of the following features. The cannula may include stainless steel, a polymer, or a combination thereof to provide atraumatic transition for gum line cannulation. The cannula may include an oval or rectangular cross-sectional shape. The bioactive glass material may include fiber, beads, or combinations thereof. The trace elements may be present at concentrations ranging from about 0.01% to about 20% by weight. The obturator may be configured to compact the bioactive glass material into periodontal pockets after delivery.

According to another aspect of the present disclosure, a method of stabilizing loose teeth is provided. The method comprises accessing a periodontal void adjacent to a loose tooth. The method comprises delivering a bioactive glass composition into the periodontal void using a delivery system, wherein the bioactive glass composition comprises boron oxide and calcium oxide as primary network formers. The method includes allowing the bioactive glass composition to provide mechanical stabilization while releasing bioactive ions for bone regeneration.

According to other aspects of the present disclosure, the method may include one or more of the following features. The bioactive glass composition may be enhanced with one or more trace elements selected from copper oxide, zinc oxide, strontium oxide, iron oxide, potassium oxide, magnesium oxide, sodium oxide, and phosphorus pentoxide at concentrations of about 0.01% to about 20% by weight. The bioactive glass composition may be configured to dissolve in interstitial fluids to create an alkaline environment. Delivering the bioactive glass composition may include manipulating an obturator of the delivery system to compact the bioactive glass composition. The bioactive glass composition may include Type I collagen at concentrations of about 0.1% to about 30% by weight. Accessing the periodontal void may comprise scaling and root planing to prepare a periodontal pocket and remove bacterial deposits.

According to another aspect of the present disclosure, a phase-invertible gel composition for periodontal therapy is provided. The phase-invertible gel composition comprises bioactive glass particles comprising boron oxide and calcium oxide as primary network formers. The composition comprises a phase-invertible carrier in water at a concentration of about 30% by weight, wherein the phase-invertible gel composition is configured to transition from liquid to gel at body temperature.

According to other aspects of the present disclosure, the phase-invertible gel composition may include one or more of the following features. The bioactive glass particles may be enhanced with trace elements at concentrations ranging from about 0.01% to about 20% by weight, wherein the trace elements may be selected from copper oxide, zinc oxide, strontium oxide, iron oxide, potassium oxide, magnesium oxide, sodium oxide, and phosphorus pentoxide. The bioactive glass particles may be added to the phase-invertible carrier at concentrations of about 10% to about 70% by weight. The composition may be prepared by compounding the phase invertible carrier at temperatures below 40° F. to form a viscous liquid with another syringe containing bioactive glass particles at temperatures below 40° F. The composition may be configured to be injected through tissue adjacent to areas of bone loss and/or periodontal voids. The phase inverted composition may be configured to retain the bioactive glass particles at treatment sites. The phase-invertible gel composition may further comprise Type I collagen at concentrations of about 0.1% to about 5% by weight.

According to another aspect of the present disclosure, a method of creating antimicrobial conditions in periodontal pockets is provided. The method comprises delivering the phase-invertible gel composition into a periodontal pocket. The method comprises allowing the phase-invertible carrier to dissolve while maintaining the bioactive glass particles in the periodontal pocket to facilitate an alkaline environment. The method comprises inhibiting periodontal pathogens via the alkaline environment.

According to another aspect of the present disclosure, a method of creating antimicrobial conditions in periodontal pockets is provided. The method may include delivering the bioactive glass material into a periodontal pocket. The method may include allowing the bioactive glass material to dissolve in interstitial fluids to facilitate an alkaline environment. The method may include inhibiting periodontal pathogens via the alkaline environment.

According to another aspect of the present disclosure, a method of treating periodontal disease is provided. The method may include preparing a periodontal pocket by scaling and root planing. The method may include inserting a delivery cannula into or adjacent the periodontal pocket. The method may include dispensing bioactive glass material from the delivery cannula into the periodontal pocket, wherein the bioactive glass material may include boron oxide and calcium oxide as primary network formers and may dissolve in interstitial fluids to form an alkaline environment configured to inhibit periodontal pathogens.

According to another aspect of the present disclosure, a boron-based bioactive glass composition for dental applications is provided. The composition may include boron oxide and calcium oxide as primary network formers and one or more trace elements present in concentrations from 0.01% to 20% by weight, wherein the trace elements may be selected from copper oxide, zinc oxide, strontium oxide, iron oxide, potassium oxide, magnesium oxide, sodium oxide, and phosphorus pentoxide, wherein the composition may be configured to dissolve in interstitial fluids to release bioactive ions that may stimulate hydroxyapatite formation.

According to another aspect of the present disclosure, a syringe-deliverable composition for periodontal therapy is provided. The composition may include spherical bioactive glass microspheres having diameters ranging from 150 to 300 microns, wherein the microspheres may include boron oxide and calcium oxide as primary network formers, and a carrier solute selected from methylcellulose-based suspensions and thixotropic formulations.

According to another aspect of the present disclosure, a compacted bioactive glass wedge for periodontal applications is provided. The wedge may include compressed bioactive glass fibers formed into a wedge shape configured to fit into periodontal voids, wherein the bioactive glass fibers may include boron oxide and calcium oxide as primary network formers and one or more trace elements selected from copper oxide, zinc oxide, strontium oxide, iron oxide, potassium oxide, magnesium oxide, sodium oxide, and phosphorus pentoxide.

According to another aspect of the present disclosure, a method of manufacturing a porous bioactive glass structure is provided. The method may include preparing a molten bioactive glass composition including boron oxide and calcium oxide as primary network formers. The method may include incorporating porogens selected from sodium chloride and polyethylene glycol into the molten bioactive glass composition. The method may include cooling the composition to form a solid structure. The method may include removing the porogens to create a porous bioactive glass structure with controlled porosity.

According to another aspect of the present disclosure, a dental implant coating composition is provided. The composition may include a boron-based bioactive glass coating applied to a surface of a dental implant, wherein the coating may include boron oxide and calcium oxide as primary network formers and one or more trace elements selected from copper oxide, zinc oxide, strontium oxide, iron oxide, potassium oxide, magnesium oxide, sodium oxide, and phosphorus pentoxide, wherein the coating may provide antimicrobial protection and may promote osseointegration.

According to another aspect of the present disclosure, a fibrous and bead bioactive glass composition is provided. The composition may include bioactive glass fibers having diameters ranging from 200 nanometers to 4000 nanometers and a length-to-width aspect ratio of at least 10, and bioactive glass microspheres having diameters ranging from 150 to 300 microns, wherein both the fibers and microspheres may include boron oxide and calcium oxide as primary network formers.

According to another aspect of the present disclosure, a method of enhancing dental implant integration is provided. The method may include placing a dental implant in a bone defect. The method may include packing bioactive glass material around the dental implant, wherein the bioactive glass material may include boron oxide and calcium oxide as primary network formers and one or more trace elements configured to release bioactive ions. The method may include allowing the bioactive glass material to stimulate bone growth around the dental implant while providing antimicrobial protection.

According to another aspect of the present disclosure, a cannula for bioactive glass delivery is provided. The cannula may include a tubular body having a rectangular cross-section with dimensions of 5.4 mm by 2.4 mm to provide a reduced insertion profile, an internal lumen configured to contain bioactive glass material, and a soft polymer tip covering a distal end of the tubular body to provide atraumatic insertion into periodontal pockets.

According to another aspect of the present disclosure, a collagen-enhanced bioactive glass composite is provided. The composite may include bioactive glass material including boron oxide and calcium oxide as primary network formers and collagen present at concentrations of 1% to 30% by weight for bone scaffolds, wherein the composite may provide structural scaffolding for cellular attachment and mineralization.

According to another aspect of the present disclosure, a kit for periodontal therapy is provided. The kit may include a handheld dispenser having a spring-loaded ejector mechanism, a plurality of pre-loaded cannula tips, wherein each tip may contain bioactive glass fiber material including boron oxide and calcium oxide as primary network formers, and instructions for delivering the bioactive glass fiber material into periodontal pockets for treating periodontal disease and stabilizing loose teeth.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A illustrates a bioactive glass composition shaped like a wedge, according to aspects of the present disclosure.

FIG. 1B illustrates a bioactive glass composition shaped like a cylinder, according to aspects of the present disclosure.

FIG. 1C illustrates a bioactive glass composition shaped like a wedge with porosity, according to aspects of the present disclosure.

FIG. 1D illustrates a hydrogel form of a bioactive glass composition, according to aspects of the present disclosure.

FIG. 2 illustrates a cross-sectional view of a tooth and surrounding periodontal structures showing the application of a wedge-shaped bioactive glass composition positioned within a periodontal pocket to address structural and biological challenges associated with periodontal tissue breakdown, according to aspects of the present disclosure.

FIG. 3A depicts a microscopic image of polymer film material at lower magnification, according to aspects of the present disclosure.

FIG. 3B depicts a microscopic image of the polymer film material of FIG. 3A at higher magnification, according to aspects of the present disclosure.

FIG. 3C depicts a microscopic image of the polymer film material of FIG. 3A showing detailed morphology, according to aspects of the present disclosure.

FIG. 4A illustrates a top view of a fiber pillow encapsulated with PLGA spray, according to aspects of the present disclosure.

FIG. 4B illustrates the fiber pillow of FIG. 4A showing web-like appearance with porosity, according to aspects of the present disclosure.

FIG. 5A shows side one of compressed bioactive glass fiber material in an initial state, according to aspects of the present disclosure.

FIG. 5B illustrates side one of the compressed bioactive glass fiber material of FIG. 5A following PLGA coating application, according to aspects of the present disclosure.

FIG. 5C shows side two of the compressed bioactive glass fiber material of FIG. 5A in an initial untreated state, according to aspects of the present disclosure.

FIG. 5D shows side two of the compressed bioactive glass fiber material of FIG. 5A following saturation treatment with PLGA solution, according to aspects of the present disclosure.

FIG. 6A illustrates a cross-sectional view of bead films, according to aspects of the present disclosure.

FIG. 6B depicts a top view of bead film under back-light illumination, according to aspects of the present disclosure.

FIG. 6C shows a top view of bead film under back-light illumination, according to aspects of the present disclosure.

FIG. 6D depicts a bottom view of bead film under back-light illumination, according to aspects of the present disclosure.

FIG. 6E depicts a cross-sectional view with first and second layers visible, according to aspects of the present disclosure.

FIG. 6F depicts the layered structures of beads, according to aspects of the present disclosure.

FIG. 6G depicts sample edges with beads, according to aspects of the present disclosure.

FIG. 6H depicts beads that may be exposed at the surface, according to aspects of the present disclosure.

FIG. 7A illustrates a cross-sectional, schematic view of a bioactive glass delivery system for periodontal therapy, according to aspects of the present disclosure.

FIG. 7B illustrates an assembled view of the bioactive glass delivery system of FIG. 7A, according to aspects of the present disclosure.

FIG. 7C illustrates a clinical application of the bioactive glass delivery system wherein a syringe may be used to deliver a bioactive glass composition into a periodontal pocket, according to aspects of the present disclosure.

FIG. 8A depicts a complete cannula assembly of a packed bioactive glass delivery system, according to aspects of the present disclosure.

FIG. 8B depicts a circular cross-sectional view of the bioactive glass delivery system of FIG. 8A, according to aspects of the present disclosure.

FIG. 8C depicts a transition region of the bioactive glass delivery system of FIG. 8A, according to aspects of the present disclosure.

FIG. 8D depicts the bioactive glass within a soft tip portion of the delivery system of FIG. 8A, according to aspects of the present disclosure.

FIG. 8E depicts a frontal perspective of a distal end of the delivery system of FIG. 8A, according to aspects of the present disclosure.

FIG. 9A illustrates a cannula with bioactive glass fiber material positioned within an internal lumen, according to aspects of the present disclosure.

FIG. 9B illustrates an initial stage of extrusion of the bioactive glass fiber material from the cannula of FIG. 9A, according to aspects of the present disclosure.

FIG. 9C illustrates continued advancement of the bioactive glass fiber material from the cannula of FIG. 9A, according to aspects of the present disclosure.

FIG. 9D illustrates further progression of the extrusion process of the bioactive glass fiber material from the cannula of FIG. 9A, according to aspects of the present disclosure.

FIG. 9E illustrates additional advancement of the bioactive glass fiber material from the cannula of FIG. 9A, according to aspects of the present disclosure.

FIG. 9F illustrates continued dispensing of the bioactive glass fiber material from the cannula of FIG. 9A, according to aspects of the present disclosure.

FIG. 9G illustrates near-complete extrusion of the bioactive glass fiber material from the cannula of FIG. 9A, according to aspects of the present disclosure.

FIG. 9H illustrates a final stage of extrusion of the bioactive glass fiber material from the cannula of FIG. 9A, according to aspects of the present disclosure.

FIG. 10 illustrates an enlarged distal end of a cannula, according to aspects of the present disclosure.

FIG. 11A depicts bioactive glass beads extruded along with fiber material, according to aspects of the present disclosure.

FIG. 11B depicts bioactive glass beads extruded along with fiber material, according to aspects of the present disclosure.

FIG. 12A depicts an oval tip cannula containing bioactive glass material, according to aspects of the present disclosure.

FIG. 12B depicts a long axis image of the oval tip cannula containing bioactive glass fiber material, according to aspects of the present disclosure.

FIG. 12C depicts a side image of the oval tip cannula containing bioactive glass fiber material, according to aspects of the present disclosure.

FIG. 12D shows a tip of the oval tip cannula containing bioactive glass fiber material, according to aspects of the present disclosure.

FIG. 12E shows a tip imaging view of the bioactive glass fiber material of FIG. 12D, according to aspects of the present disclosure.

FIG. 12F depicts a tip imaging view of the bioactive glass fiber material of FIG. 12D, according to aspects of the present disclosure.

FIG. 13A illustrates an oval tip cannula extruding a fiber and bead mix composition, according to aspects of the present disclosure.

FIG. 13B illustrates an oval tip cannula extruding fiber and bead mix composition, according to aspects of the present disclosure.

FIG. 13C illustrates an oval tip cannula extruding fiber and bead mix composition, according to aspects of the present disclosure.

FIG. 14A depicts a flat bioglass fiber positioned on a surface, according to aspects of the present disclosure.

FIG. 14B depicts the flat bioglass fiber of FIG. 14A in a folded configuration, according to aspects of the present disclosure.

FIG. 14C depicts the folded bioglass fiber of FIG. 14B being loaded into a 14GA stainless steel nozzle with a soft 40D PEBAX tip, according to aspects of the present disclosure.

FIG. 14D depicts the folded bioglass fiber of FIG. 14B in partially loaded 14GA stainless steel nozzle with a soft 40D PEBAX tip, according to aspects of the present disclosure.

FIG. 14E depicts the folded bioglass fiber of FIG. 14B in a fully loaded 14GA stainless steel nozzle with a soft 40D PEBAX tip, according to aspects of the present disclosure.

FIG. 15A depicts a syringe filled with bioactive glass beads.

FIG. 15B illustrates a syringe filled with phase-invertible carrier in liquid form.

FIG. 15C shows the formation of a phase invertible gel composition that may include the bioactive glass beads from FIG. 15A suspended in the phase invertible carrier from FIG. 15B.

FIG. 16 illustrates the clinical application of the phase invertible gel composition wherein the composition may be delivered to multiple periodontal treatment sites through syringe-based injection techniques.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Moreover, in this disclosure, relative terms, such as, for example, “about,” “substantially,” “generally,” and “approximately” are used to indicate a possible variation of ±10% of a stated value. Additionally, as used herein, the singular forms “a,” “an,” and “the” are intended to include the plural forms as well, unless the context clearly indicates otherwise. Similarly, plural terms may encompass the singular. Thus, references to “a device,” “a component,” “a system,” or the like are intended to include one or more such devices, components, systems, etc.

As used herein bioactive glass particles, bioactive glass composition, or bioactive glass materials may be used interchangeably and refer to a class of surface-reactive materials that may comprise inorganic oxides capable of forming chemical bonds with biological tissues through the formation of a biologically active hydroxyapatite layer. The bioactive glass may include network formers such as boron oxide (B₂O₃) and calcium oxide (CaO) that may provide the structural foundation for the glass matrix. The bioactive glass may undergo controlled dissolution in physiological fluids, wherein the dissolution process may release therapeutic ions including calcium, phosphate, boron, or other elements that may stimulate cellular responses and promote tissue regeneration. The bioactive glass may be formulated in various physical forms including fibers, beads, microspheres, powders, or solid structures, wherein each form may provide specific dissolution kinetics and handling characteristics suitable for different clinical applications. The bioactive glass may be enhanced with trace elements at concentrations ranging from about 0.01% to about 20% by weight to provide additional therapeutic functions such as antimicrobial activity, angiogenesis promotion, or enhanced mineralization. The bioactive glass may form strong interfacial bonds with both hard and soft tissues through the development of a silica-rich or calcium phosphate-rich surface layer that may integrate with natural tissue structures.

As used herein, Poloxamer 407 may refer to a synthetic triblock copolymer comprising a central hydrophobic chain of polyoxypropylene flanked by two hydrophilic chains of polyoxyethylene that may exhibit thermoreversible gelation properties. Poloxamer 407 may also be known by the trade names Pluronic F-127, Kolliphor P 407, and Lutrol F127 in various commercial and pharmaceutical applications. The polymer may demonstrate reverse thermal gelation behavior wherein aqueous solutions may transition from liquid states at relatively low temperatures to gel states at relatively elevated temperatures, with the phase transition temperature being dependent on polymer concentration and solution composition. Poloxamer 407 may be characterized by an average molecular weight of approximately 12,600 daltons, wherein the polyoxyethylene blocks may constitute approximately 70% of the total molecular weight while the polyoxypropylene block may constitute approximately 30% of the total molecular weight. The amphiphilic nature of Poloxamer 407 may enable the formation of micelles in aqueous solutions, wherein the hydrophobic polyoxypropylene cores may be surrounded by hydrophilic polyoxyethylene coronas that may provide solubilization capabilities for hydrophobic compounds. The thermosensitive properties of Poloxamer 407 may result from temperature-dependent changes in polymer hydration and micelle aggregation, wherein increasing temperature may lead to dehydration of the polyoxypropylene blocks and subsequent micelle packing that may create gel networks.

Periodontal disease represents a widespread condition characterized by progressive destruction of supporting structures of teeth, including gums, periodontal ligament, and alveolar bone. The condition may lead to increased tooth mobility, gum recession, and eventual tooth loss when left untreated. Periodontal disease may be caused by a combination of factors, including bacterial infection by pathogens such as Porphyromonas gingivalis, Treponema denticola, Tannerella forsythia, and Fusobacterium nucleatum, as well as host immune responses and environmental factors.

Conventional treatment methods, such as scaling and root planing, may aim to reduce calculus and bacterial loads and restore periodontal health. However, these methods may be insufficient in addressing advanced periodontal disease, such as residual bone loss, and cleanings may provide no stabilization for already loose teeth. Bone grafts, membranes, and regenerative biomaterials may conventionally be the purview of more complex and expensive oral surgeries. Physicians such as periodontal surgeons, prosthodontists, or oral and maxillofacial surgeons may typically use bone grafting materials. While effective in certain serious oral health scenarios, these professionals may often be utilized for end-stage periodontal disease or in cases of severe physiological malformations.

Dental implants may represent a widely used solution for replacing teeth lost due to periodontal disease or other causes. However, the success of dental implants may depend on adequate osseointegration, requiring healthy surrounding bone and resistance to infection. Current implant materials may focus on biocompatibility and structural integrity but may often lack additional properties, such as antimicrobial activity or the ability to actively stimulate bone regeneration. Furthermore, traditional techniques for grafting material around implants may be complex and inconsistent, particularly in cases of severe bone loss.

Bioactive glasses may have emerged as promising materials for periodontal disease therapies, bone regeneration, and enhancing dental implant outcomes due to their ability to dissolve in bodily fluids, release bioactive ions, and stimulate osteogenesis. However, most existing bioactive glass formulations may be silica-based and may lack the solubility, antimicrobial properties, and precise delivery mechanisms for periodontal and implant applications.

The present disclosure describes a boron-based bioactive glass composition that may address the limitations of conventional periodontal therapies. The bioactive glass composition may comprise boron oxide (B₂O₃) and calcium oxide (CaO) as primary network formers. These primary network formers may provide the structural foundation for the glass matrix while enabling controlled dissolution in biological environments.

The bioactive glass composition may be enhanced with one or more trace elements selected from copper oxide (CuO), zinc oxide (ZnO), strontium oxide (SrO), iron oxide (Fe₂O₃), potassium oxide (K₂O), magnesium oxide (MgO), sodium oxide (Na₂O), and phosphorus pentoxide (P₂O₅). The trace elements may be present at concentrations ranging from about 0.01% to about 20% by weight. Each trace element may contribute specific biological functions to the overall composition.

Copper oxide (CuO) may provide antimicrobial properties and may promote angiogenesis, thereby aiding in infection prevention and vascularization. Zinc oxide (ZnO) may enhance osteoblast proliferation and collagen synthesis, which may contribute to bone formation processes. Strontium oxide (SrO) may stimulate bone mineralization and may reduce osteoclast activity, potentially leading to improved bone density. Iron oxide (Fe₂O₃) may support cellular metabolism and may provide mechanical stability to the glass structure.

Calcium oxide (CaO) may be involved in hydroxyapatite formation and bone regeneration processes. Potassium oxide (K₂O) may regulate ion balance and may contribute to the dissolution profile of the glass composition. Magnesium oxide (MgO) may enhance bone cell adhesion and may promote bone healing mechanisms. Sodium oxide (Na₂O) may improve glass solubility, thereby releasing ions that may aid in osteogenesis. Phosphorus pentoxide (P₂O₅) may be involved in hydroxyapatite crystallization and bone bonding processes.

The bioactive glass composition may be combined with Type I collagen to form composite materials for various applications. For bone scaffold applications, Type I collagen may be incorporated at concentrations of about 0.1% to about 30% by weight. For soft-tissue or gum applications, Type I collagen may be incorporated at concentrations of about 1% to about 50% by weight. Type I collagen may provide a structural framework for hydroxyapatite deposition and may promote osteoblast adhesion and cellular differentiation.

The bioactive glass compositions may dissolve in interstitial fluids, creating an alkaline environment that may discourage bacterial colonization. During dissolution, the compositions may release bioactive ions that may stimulate hydroxyapatite formation and may promote osteogenesis. The dissolution process may create conditions wherein periodontal pathogens such as Porphyromonas gingivalis and Treponema denticola may experience diminished growth, as these pathogens may demonstrate reduced viability outside neutral pH ranges.

Particles of the bioactive glass composition may maintain their compositional integrity while providing controlled release of therapeutic ions over time. The combination of boron oxide and calcium oxide as primary network formers may enable predictable dissolution kinetics while maintaining structural stability during handling and placement procedures. The trace element concentrations may be adjusted to optimize specific therapeutic outcomes for different periodontal applications.

The bioactive glass composition may provide mechanical stabilization while releasing bioactive ions for bone regeneration. The physical presence of the bioactive glass material may offer immediate structural support to loose teeth by filling periodontal voids and providing mechanical reinforcement to compromised periodontal structures. Simultaneously, the dissolution process may release therapeutic ions that may stimulate cellular processes involved in bone formation and tissue regeneration.

The released bioactive ions may stimulate hydroxyapatite formation through the provision of calcium and phosphate ions that may serve as building blocks for new bone matrix. The ion release may also promote osteoblast activity and may enhance the natural bone healing response. The combination of mechanical support and biological stimulation may address both immediate stabilization needs and long-term regenerative goals in periodontal therapy.

For dental implant applications, the bioactive glass composition may be applied as coatings for dental implants to prevent infection and promote osseointegration through ion release. The coating may provide antimicrobial protection by creating localized alkaline conditions that may inhibit bacterial colonization on implant surfaces. The ion release from the coating may stimulate surrounding bone growth by providing bioactive elements that may enhance osteoblast function and bone matrix formation around the implant interface.

The bioactive glass compositions may enhance dental implant osseointegration through antimicrobial protection and stimulation of surrounding bone growth. The antimicrobial properties may reduce the risk of peri-implant infections that may compromise implant stability and long-term success. The bioactive ion release may create favorable conditions for bone cells to attach, proliferate, and form new bone tissue in direct contact with the implant surface.

Treatment durability may be optimized through sustained retention of bioactive glass particles at treatment sites and formation of stable gel matrices. When formulated as phase-invertible gel compositions, the bioactive glass may provide sustained retention of bioactive glass particles at treatment sites through formation of a stable gel matrix. The gel matrix may maintain the bioactive glass particles in position while allowing controlled dissolution and ion release over extended periods.

The gel may provide sustained retention of bioactive glass particles at treatment sites through formation of a stable gel matrix that may resist displacement by oral fluids and mechanical forces. The stable gel matrix may ensure that therapeutic concentrations of bioactive ions may be maintained in the treatment area for sufficient duration to achieve desired biological responses. The sustained retention may eliminate the need for frequent reapplication and may improve patient compliance with treatment protocols.

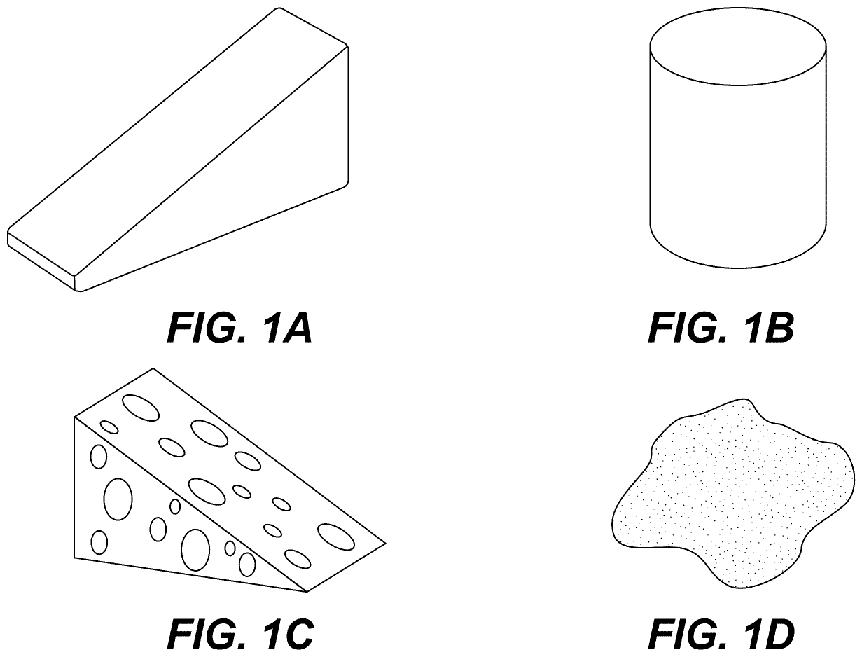

Referring to FIG. 1A, the bioactive glass composition may be formed into various wedge-shaped configurations that may be specifically designed for fitting into periodontal voids. While a triangular-shaped prism (e.g., a right-angle triangle) is depicted, the disclosure is not so limited. Rather, the wedge-shaped configuration may take any appropriate form including, but not limited to, alternative prisms, pyramids, cones, etc. The wedge-shaped form may provide a tapered geometry that may facilitate insertion into narrow periodontal spaces while maximizing contact with surrounding tissue surfaces. The wedge configuration may allow for precise placement adjacent to tooth roots wherein the bioactive glass composition may provide immediate mechanical stabilization to loose teeth while initiating dissolution processes for ion release.

The wedge-shaped (e.g., compacted sintered) bioactive glass composition may be dimensioned to match the geometry of periodontal defects, wherein the tapered profile may enable insertion into spaces that may be inaccessible to other delivery forms. The wedge shape may provide mechanical advantages during placement procedures by allowing clinicians to position the material with controlled force application. The broad base of the wedge may distribute mechanical loads across larger surface areas, which may enhance stabilization effectiveness for mobile (e.g., loose) teeth.

As shown in FIG. 1B, the bioactive glass composition may be formed into cylindrical configurations that may be tailored for insertion into alveolar sockets following tooth extraction procedures. The cylindrical form may provide uniform cross-sectional geometry that may facilitate placement into socket spaces while maintaining consistent material density throughout the volume. The cylindrical configuration may enable predictable dissolution patterns wherein ion release may occur uniformly from all surface areas.

The cylindrical bioactive glass composition may be sized to match alveolar socket dimensions, wherein the uniform diameter may provide consistent contact with socket walls. The cylindrical form may facilitate handling during placement procedures by providing a stable geometry that may resist deformation during insertion. The length of the cylindrical form may be adjusted to accommodate varying socket depths while ensuring complete filling of extraction sites.

With reference to FIG. 1C, the bioactive glass composition may be configured as porous wedge structures that may incorporate controlled porosity to enhance surface area for dissolution processes. The porous wedge structure may combine the geometric advantages of the wedge shape with increased surface area that may accelerate ion release kinetics. The porosity may be created through manufacturing processes such as porogen removal, nitrogen blasting, or laser etching techniques.

The porous wedge structure may provide enhanced dissolution characteristics wherein the increased surface area may result in more rapid ion release compared to solid forms. The porous architecture may allow interstitial fluids to penetrate throughout the material volume, which may facilitate dissolution from internal surfaces in addition to external surfaces. The porosity may be controlled to optimize the balance between mechanical stability and dissolution rate for specific clinical applications.

The porous structure may enable interstitial fluid penetration while maintaining mechanical stability for tooth stabilization applications. The interconnected pore network may provide pathways for fluid exchange that may enhance the dissolution process while preserving the structural integrity needed for mechanical support. The pore size and distribution may be tailored to achieve desired dissolution kinetics while maintaining sufficient material strength for clinical handling.

As illustrated in FIG. 1D, the bioactive glass composition may be formulated as hydrogel compositions that may be suitable for injectable applications through syringe delivery systems. The hydrogel formulation may incorporate the bioactive glass particles within a gel matrix that may provide flowable properties for injection while maintaining particle suspension. The hydrogel form may enable delivery into irregular or confined spaces that may be difficult to access with solid forms.

The hydrogel formulation may provide sustained ion release through controlled dissolution of embedded bioactive glass particles within the gel matrix. The gel carrier may maintain the bioactive glass particles in position while allowing gradual dissolution and ion diffusion to surrounding tissues. The hydrogel may be formulated with phase-invertible properties wherein the composition may transition from liquid to gel state upon exposure to body temperature conditions.

Each physical form of the bioactive glass composition may be tailored to specific clinical scenarios and delivery requirements. The wedge-shaped configurations may be selected for periodontal pocket applications wherein precise placement and mechanical stabilization may be priorities. The cylindrical forms may be chosen for alveolar socket filling applications wherein uniform material distribution may be desired. The porous wedge structures may be utilized when enhanced dissolution rates may be beneficial for rapid ion release. The hydrogel formulations may be employed when injectable delivery may be preferred for accessing confined or irregular treatment sites. It is understood that various physical forms may be used in conjunction with one another for targeted treatment or particular subject anatomy.

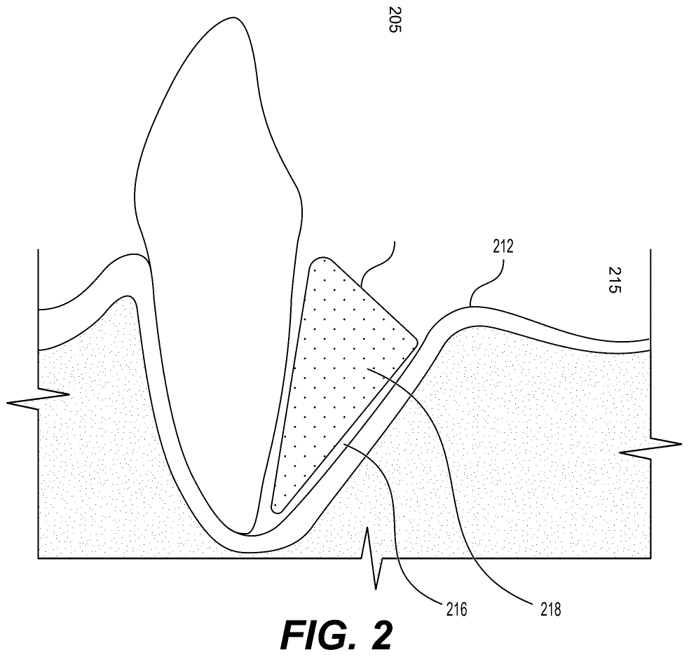

As shown in FIG. 2, the anatomical structures involved in periodontal treatment may include a tooth 205, gum (gingivae) 212, periodontal bone 215, and a periodontal pocket 216. The tooth 205 may represent the dental structure that may extend above and below the gum line and may be surrounded by supporting periodontal tissues. The tooth 205 may serve as an anchor point within the alveolar bone structure and may be affected by periodontal disease processes that may compromise tooth stability.

The gum (gingivae) 212 may comprise the soft tissue that may surround the tooth 205 and may form the visible portion of the periodontal attachment apparatus. The gum (gingivae) 212 may provide a protective barrier around the tooth and may be subject to inflammatory processes during periodontal disease progression. The periodontal bone 215 may comprise the alveolar bone structure that may normally surround and support the tooth beneath the gum (gingivae) 212. The periodontal bone 215 may provide the structural foundation for tooth retention and may be subject to resorption processes during periodontal disease progression.

The periodontal pocket 216 may represent a pathological space that may form between the tooth and the gum (gingivae) 212 due to periodontal disease processes, creating a void adjacent to the tooth. The periodontal pocket 216 may vary in depth and configuration depending on the extent and pattern of tissue breakdown. These pockets may compromise tooth stability and may provide sites for bacterial colonization that may perpetuate disease progression.

A wedge-shaped bioactive glass composition 218 may be positioned within the periodontal pocket 216 to address the structural and biological challenges associated with periodontal tissue breakdown. The wedge-shaped bioactive glass composition 218 may be delivered into the periodontal pocket 216 through any appropriate delivery mechanisms. The placement of the wedge-shaped bioactive glass composition 218 may fill the void space created by periodontal disease while providing therapeutic benefits through ion release mechanisms.

The wedge-shaped bioactive glass composition 218 may fill the periodontal pocket 216 between the tooth and the gum (gingivae) 212, thereby providing immediate mechanical support to compromised periodontal structures. The physical presence of the wedge-shaped bioactive glass composition 218 within the periodontal pocket 216 may restore structural continuity between the tooth 205 and the surrounding periodontal bone 214. This mechanical stabilization may reduce tooth mobility and may prevent further progression of periodontal breakdown.

The wedge-shaped bioactive glass composition 218 may provide mechanical stabilization while simultaneously releasing bioactive ions to promote bone regeneration within the periodontal pocket 216. The dissolution of the wedge-shaped bioactive glass composition 218 in interstitial fluids may release calcium, phosphate, and other therapeutic ions that may stimulate osteoblast activity and bone formation processes. The ion release may create favorable conditions for new bone growth that may gradually replace the wedge-shaped bioactive glass composition 218 with natural periodontal bone 215.

The positioning of the wedge-shaped bioactive glass composition 218 within the periodontal pocket 216 may enable direct contact with the tooth surface, the gum (gingivae) 212, and the existing periodontal bone 215. This contact may facilitate the formation of new periodontal tissue that may integrate with the tooth and the surrounding periodontal bone 215 structure. The wedge-shaped bioactive glass composition 218 may serve as a scaffold for tissue regeneration while providing the chemical signals needed to stimulate cellular processes involved in periodontal healing.

Manufacturing methods for creating bioactive glass structures may incorporate various techniques to optimize porosity, surface area, and dissolution characteristics for periodontal applications. These manufacturing approaches may enable the production of bioactive glass compositions with controlled physical properties that may enhance therapeutic effectiveness while maintaining structural integrity for clinical handling and placement procedures.

Liquid glass casting with porogen removal may provide a method for creating controlled porosity within bioactive glass structures. In this manufacturing process, porogens such as sodium chloride (NaCl) or high molecular weight polyethylene glycol (PEG) may be incorporated into molten bioactive glass during the casting process. The porogens may be distributed throughout the molten glass matrix, wherein they may occupy specific volumes within the glass structure as the material cools and solidifies.

Sodium chloride may serve as a porogen material that may be incorporated into the molten bioactive glass at predetermined concentrations to achieve desired porosity levels. The sodium chloride particles may maintain their crystalline structure within the cooling glass matrix, creating discrete domains of salt material distributed throughout the glass volume. Upon completion of the casting process, the sodium chloride may be removed through dissolution in aqueous solutions, leaving behind porous cavities that may correspond to the original salt particle locations and sizes.

High molecular weight polyethylene glycol with a molecular weight of 10,000 may function as an alternative porogen material for creating porosity in bioactive glass structures. The polyethylene glycol may be mixed with the molten glass wherein the polymer may form discrete phases within the glass matrix during cooling. The polyethylene glycol domains may be subsequently removed through solvent extraction processes, wherein appropriate solvents may dissolve the polymer while leaving the glass structure intact. The removal of the polyethylene glycol may create interconnected porous networks that may enhance surface area for dissolution processes.

The porogen removal process may result in bioactive glass structures with controlled porosity that may enhance dissolution rates and ion release kinetics. The porous architecture created through porogen removal may provide increased surface area for interaction with interstitial fluids, which may accelerate the dissolution process and therapeutic ion release. The porosity may also create pathways for fluid penetration throughout the material volume, enabling dissolution from internal surfaces in addition to external surfaces.

Laser etching may provide a precision manufacturing method for creating pores and channels in solid bioactive glass substrates. The laser etching process may utilize focused laser energy to selectively remove material from specific locations on the glass surface, creating controlled patterns of pores and channels. The laser parameters may be adjusted to control the depth, diameter, and spacing of the etched features, enabling customization of the surface architecture for specific applications.

The laser etching process may create precision pores and channels that may enhance dissolution rates while maintaining structural integrity of the bioactive glass substrate. The etched features may increase the surface area available for dissolution processes without compromising the overall mechanical strength of the glass structure. The precision control offered by laser etching may enable the creation of specific pore geometries that may optimize fluid flow and ion release patterns.

The channels created through laser etching may provide pathways for interstitial fluid penetration into the bioactive glass structure, which may facilitate dissolution from multiple surfaces simultaneously. The controlled geometry of the laser-etched channels may enable predictable dissolution patterns wherein ion release may occur in predetermined spatial distributions. The precision of the laser etching process may allow for the creation of complex channel networks that may optimize therapeutic ion delivery to specific tissue regions.

Nitrogen blasting may represent a manufacturing technique wherein nitrogen gas may be percolated through molten bioactive glass during the cooling process to form porous structures. The nitrogen gas may be introduced into the molten glass through controlled injection systems that may distribute gas bubbles throughout the glass volume. As the glass cools and solidifies, the nitrogen bubbles may be trapped within the matrix, creating spherical or irregular porous cavities distributed throughout the material.

The nitrogen blasting process may form porous structures with enhanced surface area for bioactivity through the creation of interconnected void networks within the glass matrix. The gas bubbles introduced during the molten state may create porous architectures that may facilitate interstitial fluid penetration and may accelerate dissolution processes. The enhanced surface area created through nitrogen blasting may increase the rate of bioactive ion release compared to solid glass structures.

The cooling process during nitrogen blasting may be controlled to optimize pore formation and distribution within the bioactive glass structure. The rate of cooling may influence the size and connectivity of the porous network, wherein slower cooling rates may allow for larger pore formation while faster cooling may result in smaller, more numerous pores. The nitrogen pressure and injection rate may be adjusted to control the overall porosity level and pore size distribution within the final glass structure.

Triangular rod assembly may provide a manufacturing method wherein bioactive glass may be cast onto ridged sheets to form triangular rods with specific dimensional characteristics. The casting process may utilize molds or forming surfaces with triangular cross-sectional profiles that may shape the molten glass into rod configurations during cooling. The triangular geometry may provide specific surface area characteristics and mechanical properties that may be advantageous for certain periodontal applications.

The triangular rods may be formed with diameters ranging from 150 to 250 micrometers, wherein the diameter may be controlled through the dimensions of the casting molds or forming surfaces. The triangular cross-sectional geometry may provide increased surface area compared to circular cross-sections of equivalent diameter, which may enhance dissolution rates and ion release kinetics. The rod geometry may also provide mechanical advantages during handling and placement procedures.

The triangular rods may be layered and compressed into mechanically stable wedge shapes or other preformed shapes such as pellets or rods through controlled assembly processes. The layering process may involve stacking multiple triangular rods in predetermined orientations to achieve desired overall geometries. The compression process may apply controlled pressure to the layered rods, creating mechanical bonds between adjacent rod surfaces while maintaining the overall structural integrity of the assembly.

The compressed assemblies of triangular rods may create mechanically stable structures that may maintain their shape during handling and placement procedures while providing controlled dissolution characteristics. The interfaces between adjacent triangular rods may create channels and void spaces that may facilitate interstitial fluid penetration throughout the assembly. These interfacial spaces may enhance the overall surface area available for dissolution processes while maintaining the mechanical stability needed for clinical applications.

Fenestrated assemblies may comprise perforated glass sheets that may be assembled into wedge shapes or other configurations for periodontal applications. The fenestrations may be created through various manufacturing processes including compression of fibrous materials, direct injection of gas bubbles during glass formation, or laser etching techniques applied to solid glass sheets. The fenestrations may provide controlled openings that may allow interstitial fluid penetration while maintaining the mechanical stability of the glass structure.

The perforated glass sheets may be assembled into wedge shapes through layering and bonding processes that may create three-dimensional structures with controlled porosity and mechanical properties. The assembly process may involve stacking multiple perforated sheets in predetermined orientations to achieve desired overall geometries while ensuring that the fenestrations may align to create continuous pathways for fluid flow. The bonding between sheets may be achieved through thermal processing, adhesive materials, or mechanical compression techniques.

The fenestrated assemblies may allow interstitial fluid penetration while maintaining mechanical stability for tooth stabilization applications. The fenestrations may provide pathways for fluid exchange that may enhance dissolution processes while preserving the structural integrity needed for mechanical support of loose teeth. The size and distribution of the fenestrations may be controlled to optimize the balance between fluid permeability and mechanical strength for specific clinical requirements.

The manufacturing methods may be selected and combined to achieve specific performance characteristics for different periodontal applications. Liquid glass casting with porogen removal may be chosen when high porosity and rapid dissolution may be desired. Laser etching may be selected when precise control of surface features may be required. Nitrogen blasting may be utilized when enhanced surface area with maintained structural integrity may be needed. Triangular rod assembly may be employed when specific mechanical properties and controlled dissolution patterns may be advantageous. Fenestrated assemblies may be created when fluid permeability combined with mechanical stability may be priorities for the intended application.

Bioactive glass fiber materials may be treated through various methods to enhance handleability while maintaining their therapeutic properties for periodontal applications. The fiber treatment processes may address the challenge that untreated bioactive glass fibers may lack sufficient structural integrity for clinical manipulation and placement procedures. These treatment methods may provide bioactive glass fiber materials with improved handling characteristics while preserving the dissolution and ion release properties that may be beneficial for periodontal therapy.

Spray coating techniques may be employed to apply polymer coatings to bioactive glass fiber materials using airbrush delivery systems. The spray coating process may utilize poly(lactic-co-glycolic acid) polymer solutions that may be prepared at specific concentrations to achieve desired coating characteristics. A poly(lactic-co-glycolic acid) polymer solution may be prepared at a concentration of 50 mg/ml in acetone to provide appropriate viscosity and coating properties for airbrush application.

The airbrush systems may be configured with appropriate nozzle sizes and pressure settings to achieve controlled spray patterns for coating bioactive glass fiber substrates. The airbrush application may utilize a 0.5 mm nozzle operating at low pressure settings to minimize turbulence that may displace the fiber materials during coating procedures. The spray coating process may be applied to both top and bottom surfaces of bioactive glass fiber materials to ensure comprehensive coverage and coating uniformity.

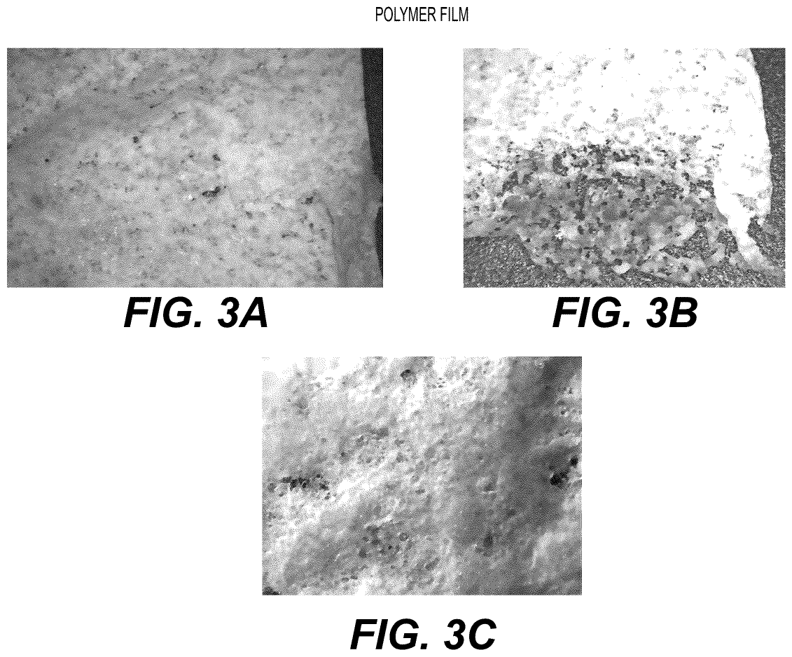

Referring to FIG. 3A, FIG. 3B, and FIG. 3C, the spray coating process may create polymer films with distinctive structural characteristics on bioactive glass fiber substrates. The polymer film material may exhibit a porous, web-like network structure with interconnected strands distributed throughout the coating layer. The web-like appearance may result from the spray application process wherein the polymer solution may form continuous strands that may interconnect across the fiber surface during solvent evaporation.

The interconnected strands created through spray coating may form irregular openings and channels within the coating structure that may allow for interstitial fluid penetration while providing structural integrity to the fiber material. The porous network may maintain pathways for fluid exchange that may be necessary for dissolution processes while creating sufficient mechanical bonding to hold individual fibers together. The strand network may create a three-dimensional structure that may encapsulate the fiber material while preserving access for biological fluids.

The spray coating technique may result in a porous shell coating on the surface of bioactive glass fiber materials that may provide handleability improvements without completely encasing the fibers. The porous shell may allow the underlying bioactive glass fibers to remain accessible to interstitial fluids while providing sufficient structural support for clinical manipulation. The shell coating may create a network of polymer strands that may interconnect across the fiber surface, creating channels and openings that may facilitate fluid penetration.

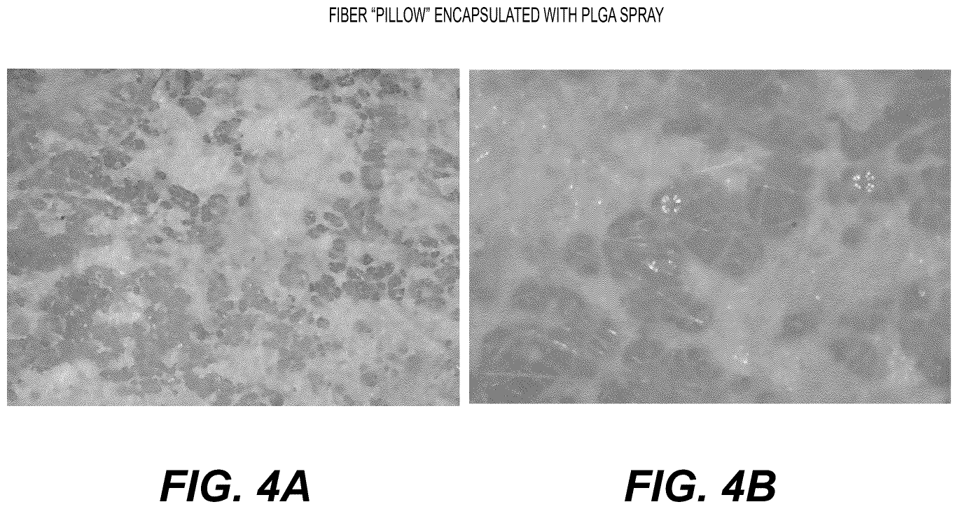

As illustrated in FIG. 4A and FIG. 4B, fiber pillow configurations may be created through spray coating applications wherein bioactive glass fiber materials may be formed into three-dimensional pillow structures that may be encapsulated with poly(lactic-co-glycolic acid) spray coatings. The fiber pillow may maintain its three-dimensional structure while the coating may provide a degree of structural integrity to the otherwise loose fiber material. The spray coating may create variable coverage across the pillow surface, with some regions showing more complete coating while other areas may display minimal coverage.

The fiber pillow encapsulated with poly(lactic-co-glycolic acid) spray coating may exhibit a web-like appearance with significant porosity throughout the structure. The coating may create a porous shell on the surface of the pillow wherein glass fibers may be visible beneath the surface coating in areas with lighter coverage. The porosity of the coating may depend on the amount of coating material applied, wherein heavier coating applications may result in more complete surface coverage while lighter applications may maintain greater porosity.

The spray coating process may provide variable coating thickness and coverage that may be controlled through application parameters such as spray duration, nozzle distance, and solution concentration. The variability in coating coverage may be advantageous for certain applications wherein complete encapsulation may not be desired. The partial coating may allow direct contact between bioactive glass fibers and biological fluids while providing sufficient structural support for handling and placement procedures.

Compression molding may represent an alternative method for enhancing the handleability of bioactive glass fiber materials through mechanical processing techniques. The compression molding process may involve placing bioactive glass fiber material between silicone mats and applying controlled pressure to create compressed fiber sheets with improved structural characteristics. The compression process may be performed without spacers to control thickness, allowing the natural compressibility of the fiber material to determine the final thickness of the compressed sheet.

The bioactive glass fiber material may be easily compressible under applied pressure and may maintain its compressed shape after pressure removal. The compression process may create mechanical bonding between adjacent fibers through contact pressure and potential surface interactions. The compressed fiber material may exhibit improved handleability compared to uncompressed fiber materials while retaining the porous structure that may be necessary for dissolution and ion release processes.

The compressed bioactive glass fiber sheets may lack sufficient handleability for clinical applications in their untreated state, wherein the compressed material may fall apart when attempts are made to lift the sheets from the compression surfaces. The compressed fiber material may require additional treatment to achieve the structural integrity needed for clinical manipulation and placement procedures. The compression process alone may not provide adequate bonding between fibers to create a handleable material.

Saturation treatment with poly(lactic-co-glycolic acid) solution may be applied to compressed bioactive glass fiber sheets to provide enhanced handleability characteristics. The saturation process may involve applying poly(lactic-co-glycolic acid) solution at a concentration of 50 mg/ml in acetone to the compressed fiber material until the solution penetrates throughout the fiber matrix. The saturation treatment may create polymer bonding between adjacent fibers while maintaining the porous structure of the compressed material.

The bioactive glass fiber material may be saturated with poly(lactic-co-glycolic acid) solution at a concentration of 50 mg/ml in acetone to provide handleability for clinical applications. The saturation process may involve applying the polymer solution to the compressed fiber material in sufficient quantity to penetrate throughout the fiber matrix while avoiding excessive polymer loading that may compromise dissolution characteristics. The acetone solvent may facilitate penetration of the polymer solution into the fiber structure and may subsequently evaporate to leave polymer bonding between fibers.

The saturated bioactive glass fiber material may be lifted and cut without concern for fiber loss or structural breakdown. The polymer saturation may create sufficient bonding between individual fibers to enable the compressed sheets to be handled as coherent materials during clinical procedures. The saturated material may be cut into specific shapes and sizes that may be appropriate for different periodontal applications while maintaining structural integrity during manipulation.

The saturation treatment may create handleable bioactive glass fiber materials that may be inserted into periodontal pockets without losing fibers during placement procedures. The polymer bonding created through saturation may prevent individual fibers from separating from the main material during insertion and positioning within periodontal defects. The handleability enhancement may enable clinicians to manipulate the bioactive glass fiber material with confidence during placement procedures while ensuring that the material may remain intact within the treatment site.

The combination of compression molding and polymer saturation may create bioactive glass fiber materials with optimized handling characteristics for periodontal applications. The compression process may create thin sheets with controlled thickness and density while the polymer saturation may provide the structural integrity needed for clinical manipulation. The dual treatment approach may preserve the porous structure and dissolution characteristics of the bioactive glass fibers while enabling practical clinical use of the material.

The handleability enhancement methods may be selected based on specific clinical requirements and application preferences. Spray coating techniques may be chosen when partial coating coverage may be desired to maintain maximum fiber accessibility while providing minimal structural support. Compression molding with polymer saturation may be selected when thin, handleable sheets may be required for insertion into confined periodontal spaces. Both methods may preserve the therapeutic properties of the bioactive glass fibers while addressing the practical challenges associated with clinical manipulation and placement of fibrous materials.

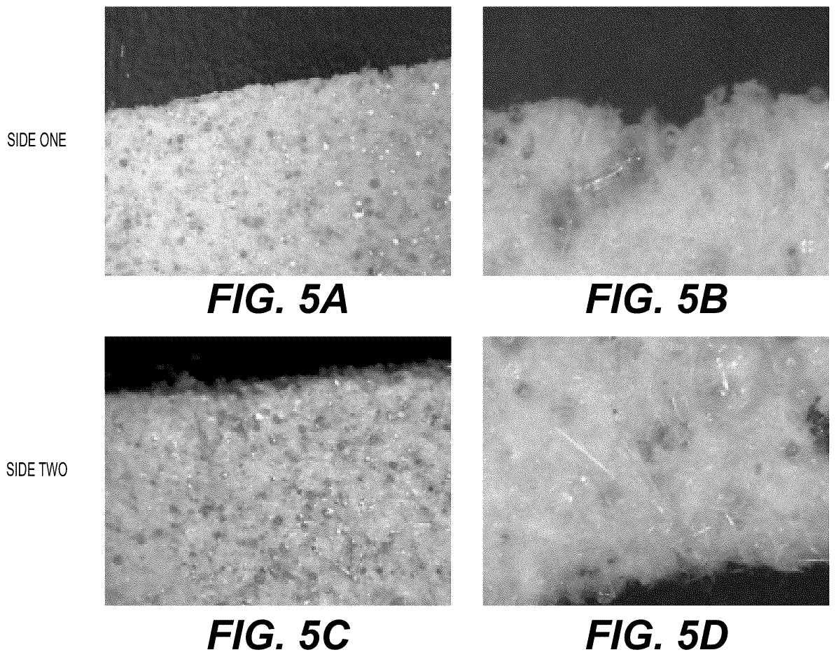

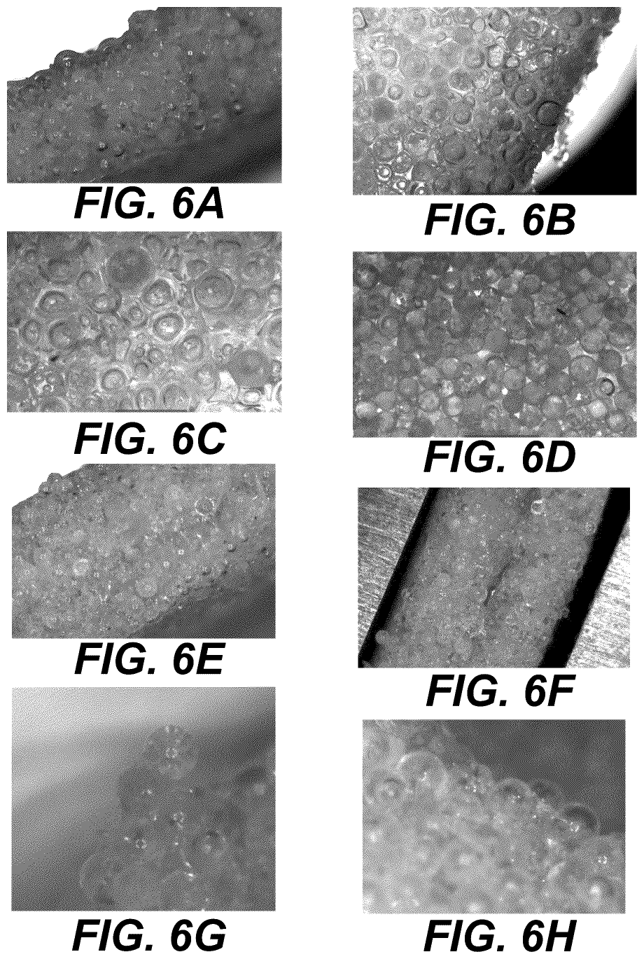

Referring to FIG. 5A, FIG. 5B, FIG. 5C, and FIG. 5D, the manufacturing and coating process for compressed bioactive glass fiber material is illustrated through multiple stages of processing and treatment. The figures demonstrate the progression from initial compression through various coating applications to achieve handleable bioactive glass compositions suitable for periodontal applications, wherein FIG. 5A and FIG. 5B show side 1 of the material and FIG. 5C and FIG. 5D show side 2 of the material.

FIG. 5A shows side 1 of the compressed bioactive glass fiber material in its initial state following compression between silicone mats using a bench scale press. The bioactive glass fiber material may be easily compressible under applied pressure and may maintain its compressed shape after pressure removal. The compressed material may exhibit a fibrous structure with individual fibers visible throughout the composition. The initial compressed state may demonstrate good shape retention characteristics wherein the material may hold its compressed form without immediate expansion or structural relaxation.

The compressed bioactive glass fiber material in its untreated state may exhibit poor handleability characteristics wherein the fabric may fall apart when attempts may be made to lift the material from surfaces. The lack of sufficient bonding between individual fibers may result in structural disintegration during handling procedures. The compressed material may require additional treatment to achieve the structural integrity needed for clinical manipulation and placement into periodontal pockets.

FIG. 5B illustrates side 1 of the compressed bioactive glass fiber material following initial PLGA coating application through spray techniques. The spray coating process may involve applying poly(lactic-co-glycolic acid) solution to one surface of the compressed fiber material to provide structural reinforcement. The spray coating application may enable the separation of the material from the silicone mat surface by creating polymer bonding between fibers on the coated surface.

The spray-coated material may demonstrate improved handleability on the coated surface wherein the PLGA polymer may create sufficient bonding to enable removal from the silicone mat. The opposite side of the material that may not receive spray coating may exhibit cracking and folding characteristics during handling procedures. The single-sided spray coating approach may provide insufficient structural integrity for clinical applications wherein the uncoated surface may remain prone to structural failure during manipulation.

FIG. 5C shows side 2 of the compressed bioactive glass fiber material in its initial untreated state following compression between silicone mats. The untreated side 2 may exhibit similar structural characteristics to the initial compressed state of side 1, wherein individual fibers may be visible throughout the composition and the material may maintain its compressed form. The surface may display a fibrous network structure with fibers arranged in various orientations throughout the compressed matrix.

The untreated side 2 may demonstrate the same poor handleability characteristics as the initial compressed material wherein the lack of polymer bonding between fibers may result in structural weakness during manipulation attempts. The surface may show the natural arrangement of compressed fibers without the reinforcement provided by PLGA coating, wherein the fibers may remain loosely bonded through compression forces alone. The untreated condition of side 2 may highlight the need for additional treatment to achieve sufficient structural integrity for clinical applications.

FIG. 5D shows side 2 of the compressed bioactive glass fiber material following saturation treatment with PLGA solution wherein the material may be saturated with poly(lactic-co-glycolic acid) solution at a concentration of 50 mg/ml in acetone. The saturation process may involve applying the polymer solution to the compressed fiber material in sufficient quantity to penetrate throughout the fiber matrix. The solution saturation approach may provide superior handleability characteristics compared to spray coating techniques by creating polymer bonding throughout the material volume rather than only at surface regions.

The surface views of FIGS. 5A-5D show compressed bioactive glass fiber material with visible individual fibers and small spherical particles distributed across surfaces, demonstrating the heterogeneous nature of the bioactive glass composition materials. Dense arrangements of spherical glass beads or microspheres with varying sizes may create packed structures with visible interstitial spaces that may facilitate fluid penetration and dissolution processes. The spherical particles may show rounded morphology and size distribution within matrix materials that may influence release kinetics.

The layered bioactive glass compositions may show material thickness and surface characteristics that may result from compression or assembly processes. The structural features may be visible at different magnifications, revealing porosity and organizational patterns that may affect therapeutic performance. The integration of different glass forms may be demonstrated wherein spherical particles may be embedded within or attached to substrate materials, showing how multiple morphologies may be combined to achieve desired therapeutic characteristics.

The saturated bioactive glass fiber material may demonstrate improved handleability characteristics wherein the material may be lifted and cut without concern for fiber loss or structural breakdown. The saturation treatment may create sufficient bonding between individual fibers to enable the compressed sheets to be handled as coherent materials during clinical procedures. The material may be cut into specific shapes and sizes that may be appropriate for different periodontal applications while maintaining structural integrity during manipulation.

The solution saturation method may work very well for creating handleable bioactive glass fiber materials that may be suitable for insertion into periodontal pockets. The saturated material may maintain its structural integrity during placement procedures wherein individual fibers may remain bonded within the compressed matrix. The saturation approach may provide advantages over spray coating techniques by ensuring uniform polymer distribution throughout the material thickness rather than concentrating polymer at surface regions.

In other embodiments, the saturation process may involve adding more PLGA to the solution to further enhance handling characteristics and structural stability. The increased polymer content may provide additional bonding between fibers while maintaining the porous structure needed for dissolution and ion release processes. The optimization of PLGA concentration may balance the competing requirements of handleability and therapeutic effectiveness wherein sufficient polymer may be present to enable clinical manipulation while avoiding excessive polymer loading that may compromise dissolution characteristics.

The progression from untreated compressed material through spray coating to solution saturation may demonstrate the importance of polymer treatment for achieving handleable bioactive glass fiber compositions. The saturation approach may provide the most effective method for creating materials that may combine the therapeutic benefits of bioactive glass fibers with the structural integrity needed for clinical applications. The handleable fiber materials created through saturation treatment may enable practical use of bioactive glass fibers in periodontal therapy applications wherein the materials may be manipulated and placed with confidence during clinical procedures.

The compressed bioactive glass fiber material with PLGA treatment may be cut into small pieces of various sizes and geometries to accommodate different periodontal application requirements. The cutting process may be performed using standard surgical instruments such as scissors or scalpels wherein the PLGA-treated material may maintain its structural integrity during cutting operations. The wetting of the material during PLGA treatment may make the material more flexible, thereby facilitating the cutting process and enabling easier manipulation during placement procedures. The ability to cut the material into customized shapes may enable clinicians to prepare bioactive glass pieces that may precisely match the dimensions and configurations of specific periodontal defects. The cut pieces may retain their handleability characteristics wherein the PLGA bonding may prevent fiber separation along cut edges. In cases wherein enhanced handling characteristics may be desired, the PLGA concentration in the saturation solution may be increased to provide additional structural reinforcement. The increased polymer content may create stronger bonding between individual fibers while maintaining the porous architecture needed for therapeutic ion release. The optimization of PLGA concentration may be adjusted based on specific clinical handling requirements wherein higher concentrations may provide improved structural stability for complex manipulation procedures while lower concentrations may be sufficient for straightforward placement applications.

Referring to FIGS. 6A-6H, various views of PLGA (poly(lactic-co-glycolic acid)) bead and fiber film compositions are presented at different magnifications and processing stages, showing the diversity of forms that may be achieved through different manufacturing and processing techniques. The PLGA compositions may be prepared using PLGA from Polyscience (23987) that may be dissolved in anhydrous acetone (Sigma 179124) at a concentration of 50 mg/ml to create a coating solution. The PLGA solution may be applied to both bioactive glass beads and fiber materials through controlled coating processes that may create composite structures with enhanced handling characteristics and controlled dissolution properties.

The fiber coating process may involve cutting bioactive glass fiber material into approximately 1-inch cubes that may be placed into aluminum pans wherein the PLGA solution may be dripped onto the fiber until saturation is achieved. The fiber samples may be prepared with varying solution ratios, wherein about 0.42 g of fiber may be combined with about 0.66 g of PLGA solution or about 0.64 g of fiber may be combined with about 2.1 g of solution to achieve different coating densities. The bead coating process may involve placing bioactive glass beads into Teflon-coated muffin pans wherein the PLGA solution may be added and mixed to ensure uniform dispersion of the beads throughout the polymer matrix. The bead compositions may be prepared with 2.49 g of beads combined with 7 g of solution to create thin samples measuring approximately 1.2 mm in thickness, or 3.22 g of beads combined with 9 g of solution to create thick samples measuring approximately 1.5 mm in thickness.

FIG. 6A illustrates a cross-sectional view of bead films showing 6-7 bead layers wherein a polymer film comprising approximately 50% of the bead thickness may be visible on the top surface while no coating may be identified on beads in the middle or bottom surface regions. FIG. 6B depicts a top view of bead film under back-light illumination at the edge wherein the film thickness may correspond to approximately 50% of the bead thickness on the top surface. FIG. 6C shows a top view of bead film under back-light illumination at the center wherein the polymer film may maintain consistent thickness across the surface. FIG. 6D illustrates a bottom view of bead film under back-light illumination wherein the lack of coating on the bottom surface may be evident, and the beads may be organized in regular patterns with some gaps present wherein beads may have been lost during processing. FIGS. 6E-6H demonstrate back-to-back sample configurations that may be created by cutting 7 mm×28 mm strips from sample 50-D, cutting the strips in half, saturating the top surfaces with anhydrous acetone, and pressing the pieces together until adhesion is achieved. The resulting samples may measure approximately 7 mm×14 mm with a thickness of 2.5 mm, wherein FIG. 6E shows a cross-sectional view with first and second layers visible, FIG. 6F depicts the layered structure, FIG. 6G illustrates sample edges with beads suspended by polymer attachments and polymer residue, and FIG. 6H shows beads that may be exposed at the surface.

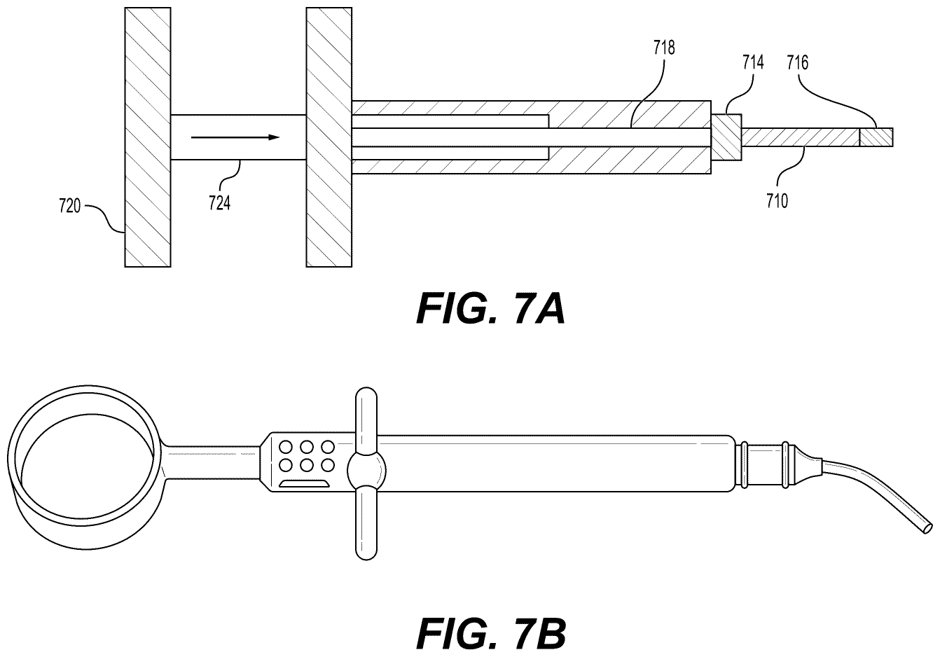

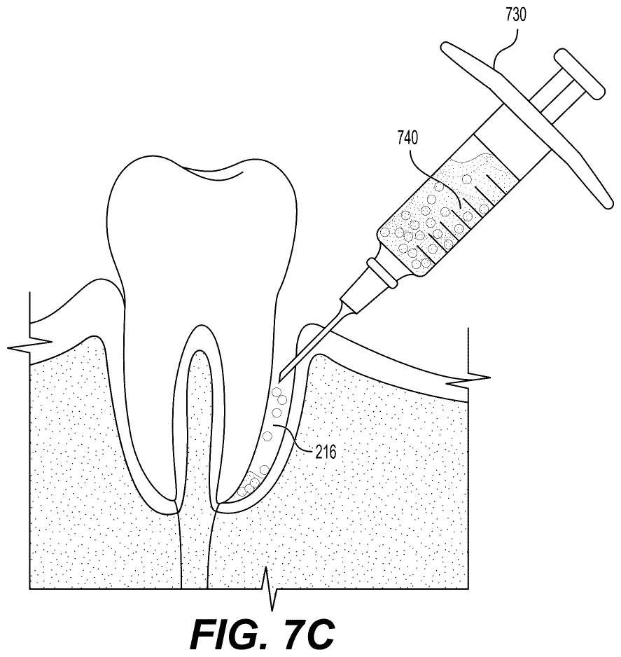

Referring to FIG. 7A and FIG. 7B, a bioactive glass delivery system for periodontal therapy may comprise a cannula having a proximal end and a distal end with an internal lumen extending therebetween. The delivery system may enable controlled dispensing of bioactive glass fiber material into periodontal defects through a precision delivery mechanism that may facilitate accurate placement of therapeutic materials. The cannula may provide a conduit for bioactive glass fiber material wherein the internal lumen may contain and guide the material during delivery procedures.

As shown in FIG. 7A, a loading cannula 710 may have an internal lumen configured to contain the bioactive glass composition. The loading cannula 710 may provide the primary structural component of the delivery system wherein the internal lumen may extend from the proximal end to the distal end to create a continuous pathway for material delivery. The internal lumen dimensions may be sized to accommodate the bioactive glass fiber material while enabling controlled advancement through the cannula structure.

A nozzle 716 may be positioned at the distal end of the loading cannula 710. The nozzle 716 may be tapered so as to provide a tapered outlet for controlled delivery of the bioactive glass composition. The nozzle 716 may feature a reduced diameter at its distal tip to facilitate insertion into periodontal pockets and controlled dispensing of the bioactive glass material. The tapered configuration of the nozzle 716 may enable precise placement of the bioactive glass composition into confined periodontal spaces while minimizing trauma to surrounding tissues.

A connector 714 may be positioned at the proximal end of the loading cannula 710, providing an interface for attachment to a dispensing mechanism. The connector 714 may enable secure coupling between the loading cannula 710 and external delivery systems that may provide the force needed to advance bioactive glass material through the internal lumen. The connector 714 may be configured to accept various types of dispensing mechanisms while maintaining a sealed interface that may prevent material leakage during delivery procedures.

A plunger assembly may be provided comprising a plunger body 720 connected to a plunger pin 718 via a connector 724. The plunger pin 718 may be configured to be inserted into the internal lumen of the loading cannula 710 through the connector 714, enabling advancement of the bioactive glass composition through the nozzle 716. The plunger pin 718 may serve as an obturator positioned within the internal lumen and configured to advance the bioactive glass fiber material through the distal end of the cannula.

The plunger body 720 may provide a surface for applying force to drive the plunger pin 716 distally, thereby expelling the bioactive glass composition from the nozzle. The plunger body 720 may be dimensioned to enable manual operation wherein clinicians may apply controlled pressure to advance the bioactive glass material at desired rates and may take any appropriate shape such as a generally planar element (see FIG. 7A) or a finger or thumb ring (see FIG. 7B). The plunger body 720 may provide mechanical advantage that may enable precise control over material delivery rates and quantities.

The bioactive glass fiber material loaded (e.g., preloaded) within the internal lumen which may have diameters ranging from about 200 nm to about 4000 nm and a length-to-width aspect ratio of at least 10. The fiber dimensions may provide high surface area for ion release while maintaining structural characteristics that may enable delivery through the cannula system. The length-to-width aspect ratio may ensure that the fibers may maintain their fibrous morphology during delivery procedures while providing enhanced dissolution characteristics compared to spherical particles.

The cannula may be manufactured from polymer materials selected from polytetrafluoroethylene, high-density polyethylene, low-density polyethylene, or polyether block amide. These polymer materials may provide biocompatibility, flexibility, and chemical resistance properties that may be suitable for periodontal applications. Polytetrafluoroethylene may offer low friction characteristics that may facilitate smooth advancement of bioactive glass material through the internal lumen. High-density polyethylene and low-density polyethylene may provide varying degrees of flexibility and strength characteristics that may be selected based on specific application requirements.

Polyether block amide may provide enhanced flexibility and softness characteristics that may be advantageous for patient comfort during periodontal procedures. The polymer material selection may influence the handling characteristics of the cannula wherein softer materials may provide improved patient comfort while harder materials may offer enhanced structural stability during delivery procedures. The polymer materials may be selected to provide atraumatic insertion characteristics while maintaining sufficient structural integrity for controlled material delivery.

The cannula may be formed entirely of stainless steel to provide structural strength and dimensional stability that may enable precise control over material delivery. The stainless steel construction may provide durability and resistance to deformation during clinical use while maintaining appropriate dimensional tolerances for accurate material dispensing. The stainless steel material may be selected to provide biocompatibility characteristics suitable for periodontal applications.

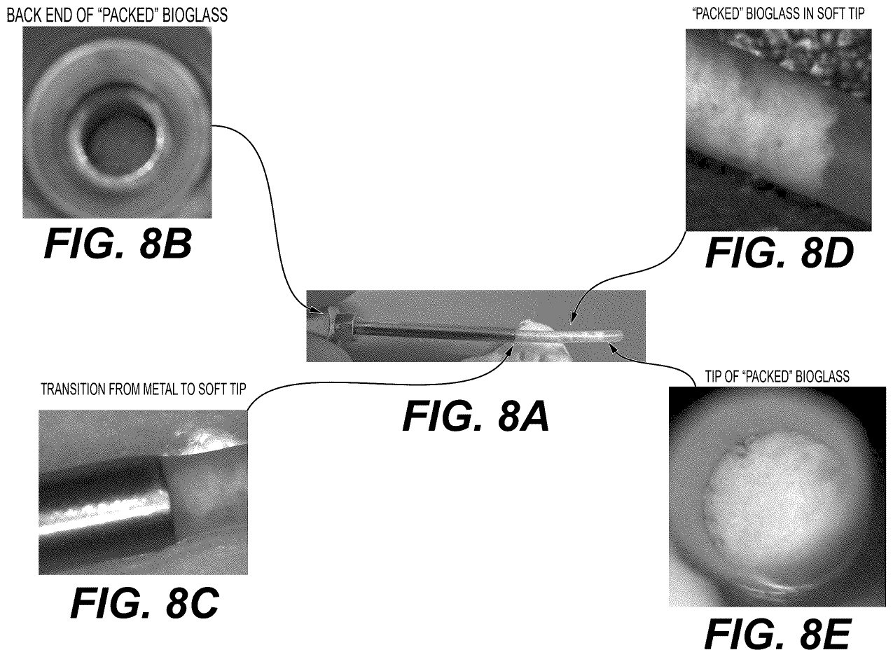

The cannula may may be constructed with part stainless steel and part polymer (as shown in FIGS. 8A-8E) to provide atraumatic transition for gum line cannulation. The stainless steel portion may provide structural strength and dimensional stability while the polymer portion may minimize tissue trauma during insertion procedures. The polymer portion may be positioned at the distal end of the cannula to create a smooth, soft interface with periodontal tissues, wherein the combination of stainless steel structural support with polymer surface characteristics may optimize both delivery precision and patient comfort during periodontal treatment procedures.

The cannula may have an oval or rectangular cross-sectional shape to increase loading density while reducing the profile needed to enter dental pockets. The oval cross-sectional configuration may provide increased internal volume compared to circular cross-sections of equivalent maximum dimension, thereby enabling greater quantities of bioactive glass material to be loaded within the cannula. The rectangular cross-sectional shape may further optimize the volume-to-profile ratio by maximizing internal area while minimizing the insertion profile required for periodontal access.

The oval or rectangular cross-sectional shapes may reduce the profile needed to enter dental pockets by minimizing the maximum dimension that may contact periodontal tissues during insertion. The reduced profile may enable access to confined periodontal spaces that may be inaccessible to larger diameter circular cannulas. The optimized cross-sectional geometry may enhance the clinical utility of the delivery system by expanding the range of periodontal sites that may be treated with the bioactive glass material.

The nozzle 716 may have curved configurations (see FIG. 7B) with flexible obturators for accessing difficult periodontal spaces. The curved nozzle configuration may enable delivery of bioactive glass material into periodontal sites that may be positioned at angles relative to the approach path of the delivery system. The flexible obturator may bend to follow the curved path of the nozzle while maintaining the ability to advance bioactive glass material through the delivery system.

The loading cannula 710 may be manufactured with varying softness levels for patient comfort or deformation of tip into periodontal space. The softness characteristics may be controlled through material selection and processing parameters to achieve desired flexibility and deformation properties. Softer cannula materials may provide enhanced patient comfort by reducing insertion forces and tissue trauma while enabling the tip to deform and conform to irregular periodontal geometries.

The delivery system may be operated using a screw drive mechanism to advance the obturator for precise delivery control. The screw drive mechanism may provide mechanical advantage that may enable fine control over the advancement rate of the plunger pin 718 through the loading cannula 710. The threaded advancement system may allow clinicians to control material delivery rates with greater precision compared to direct manual pressure application.

The nozzle may be manufactured from materials similar to medical device insertion sheaths including high-density polyethylene, low-density polyethylene, or polyether block amide. These materials may provide the flexibility and biocompatibility characteristics that may be appropriate for periodontal applications while offering manufacturing advantages for creating complex nozzle geometries. The material selection may influence the insertion characteristics and patient comfort during periodontal procedures.

The loading cannula 710 may have an outer diameter reduced to about 4 French or about 1.35 mm with built-in obturator of approximately 0.95 mm diameter. The miniaturized dimensions may enable access to confined periodontal spaces while maintaining sufficient internal volume for bioactive glass material loading. The about 4 French outer diameter may provide a low-profile insertion characteristic that may minimize tissue trauma during periodontal access procedures.

The built-in obturator diameter of approximately 0.95 mm may provide appropriate clearance within the about 1.35 mm internal diameter of the loading cannula 710 while enabling effective advancement of bioactive glass material. The dimensional relationship between the obturator and cannula may ensure smooth operation while preventing material bypass around the obturator during delivery procedures. The miniaturized dimensions may expand the range of periodontal sites that may be accessible for bioactive glass delivery.

Referring to FIG. 7C, a clinical application of the bioactive glass delivery system may be illustrated wherein a syringe 730 filled with bioactive glass composition 740 may be used to fill a periodontal pocket 216 with bioactive glass composition 740. The syringe 730 may provide an alternative delivery mechanism that may enable controlled dispensing of bioactive glass material into periodontal defects through conventional injection techniques. The periodontal pocket 216 may represent a pathological space that may have formed between the tooth and surrounding periodontal tissues due to disease processes, creating a void that may require therapeutic intervention.

Referring to FIG. 8A, FIG. 8B, FIG. 8C, FIG. 8D, and FIG. 8E, the packed bioactive glass in soft tip configuration is illustrated showing multiple detailed views of the delivery system. FIG. 8A shows a complete cannula assembly wherein the bioactive glass material is visible within the transparent soft tip section. The soft tip configuration may provide atraumatic insertion characteristics while maintaining the structural integrity needed to deliver the compressed bioactive glass fiber material.

FIG. 8B depicts a circular cross-sectional view, indicating the compressed nature of the bioactive glass material within the cannula. The layered or stratified appearance may result from the compression process wherein individual fibers may be organized into distinct layers within the cannula volume. The compressed structure may provide controlled density characteristics that may influence dissolution and ion release patterns.

FIG. 8C depicts the interface region wherein the bioactive glass fiber material changes from the rigid cannula section to the flexible tip section. The bioactive glass material may maintains its compressed structure through this transition while adapting to the changing internal geometry of the delivery system. The transition region is designed to prevent material binding or obstruction during advancement through the delivery system.

FIG. 8D depicts the bioactive glass fiber material within the soft tip portion of the delivery system wherein the compressed fiber structure may be visible through the translucent tip material. The soft tip section may encapsulate the bioactive glass material while providing flexibility for atraumatic insertion into periodontal pockets. The fiber material may maintain its compressed configuration within the soft tip wherein the individual fibers may remain organized in a dense arrangement that may facilitate controlled delivery. The soft tip material may provide a protective barrier around the bioactive glass fibers while enabling the material to conform to irregular periodontal geometries during insertion procedures.

FIG. 8E depicts a frontal perspective of the distal end wherein the compressed bioactive glass fiber material may be positioned for delivery into periodontal sites. The circular opening may reveal the compressed bioactive glass fiber material in its final delivery configuration. The tip configuration may enable controlled dispensing of the bioactive glass material while providing visual confirmation of material positioning within the delivery system.

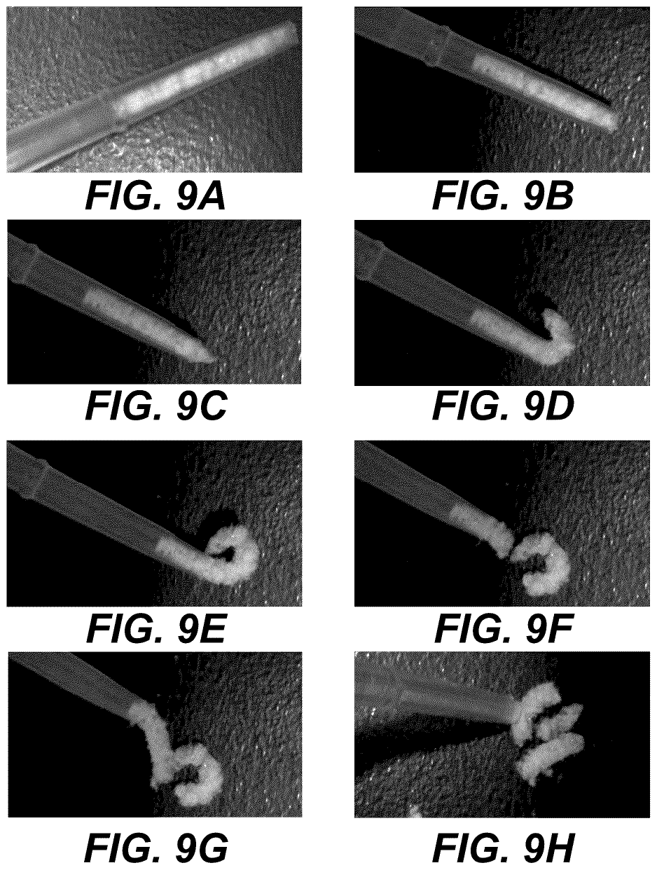

Referring to FIGS. 9A-9H, the sequential process of bioactive glass fiber material being extruded from the delivery cannula is illustrated as the obturator advances distally through the internal lumen. The sequence may demonstrate the progressive dispensing of the bioactive glass composition through the distal end of the cannula as controlled pressure is applied to the plunger assembly. The sequential images show the controlled advancement of bioactive glass material from initial positioning within the cannula to complete extrusion at the distal end.

FIG. 9A shows the cannula with bioactive glass fiber material visible within the internal lumen near the distal tip, representing the initial loaded configuration wherein the material may be positioned for delivery. FIG. 9B shows an initial stage of extrusion wherein a relatively small amount of bioactive glass fiber material may begin to emerge from the distal end of the cannula as the obturator advances. The initial emergence may demonstrate the controlled nature of the delivery process wherein material may be dispensed in measured quantities.

FIG. 9C depicts continued advancement of the bioactive glass fiber material with an increased amount of material extending beyond the cannula tip. FIG. 9D illustrates further progression of the extrusion process with the bioactive glass fiber material forming a more substantial protrusion from the distal end. The progressive advancement may demonstrate the controlled delivery characteristics wherein the rate of material dispensing may be regulated through obturator advancement speed.

FIG. 9E demonstrates additional advancement showing the extruded bioactive glass fiber material extending further from the cannula and beginning to accumulate at the tip. FIG. 9F depicts continued dispensing with the bioactive glass fiber material forming a larger mass at the distal end of the cannula. The accumulation process shows how the bioactive glass material may maintain its structural integrity during extrusion while forming a deliverable mass at the cannula tip.

FIG. 9G illustrates near-complete extrusion with a substantial volume of bioactive glass fiber material dispensed from the cannula and accumulated at the tip. FIG. 9H illustrates the final stage of the extrusion sequence showing the maximum amount of bioactive glass fiber material dispensed from the cannula, forming a compressed mass at the distal end. The final extrusion stage demonstrate the complete delivery of the loaded bioactive glass material from the cannula system.

The obturator may become flush with the distal end of the cannula after complete extrusion and may be used for compaction of the bioactive glass material into the periodontal void. When the bioactive glass material has been completely extruded, the obturator may advance to the distal opening of the cannula wherein the obturator tip may be positioned for direct contact with the delivered material. The flush positioning may enable the obturator to function as a compaction tool that may be used to press the bioactive glass material into periodontal defects.

The compaction capability provided by the flush obturator positioning may enable clinicians to apply controlled pressure to the delivered bioactive glass material to ensure proper placement within periodontal voids. The obturator may serve a dual function as both a delivery mechanism during the extrusion process and a compaction tool during the final placement procedures. The compaction process may optimize the contact between the bioactive glass material and periodontal tissues while ensuring complete filling of defect spaces.

The delivery system may provide a mechanism for controlled dispensing of bioactive glass material into periodontal defects through the coordinated operation of the cannula, obturator, and plunger assembly components. The system may enable precise placement of therapeutic materials while minimizing procedural complexity and patient discomfort. The controlled delivery and compaction capabilities may enhance the clinical effectiveness of bioactive glass therapy for periodontal applications by ensuring optimal material placement and tissue contact.

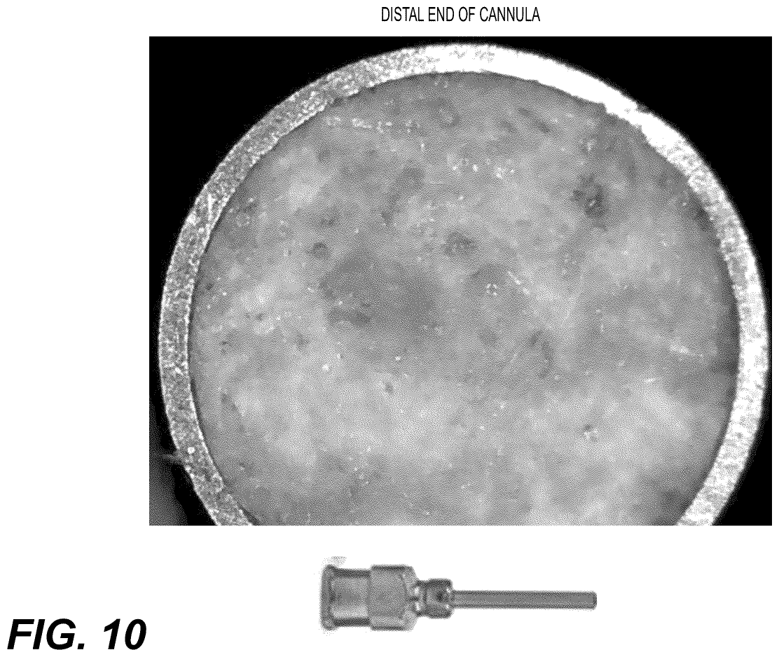

Referring to FIG. 10, an enlarged distal end of a cannula is shown with bioactive glass composition positioned within the delivery system. The cannula may comprise a delivery opening wherein the bioactive glass composition may be visible through the distal aperture. The bioactive glass composition may exhibit a disc-shaped configuration with a granular structure that may be distributed throughout the material matrix. The composition may display a textured surface with particles or domains that may be arranged across the material body.

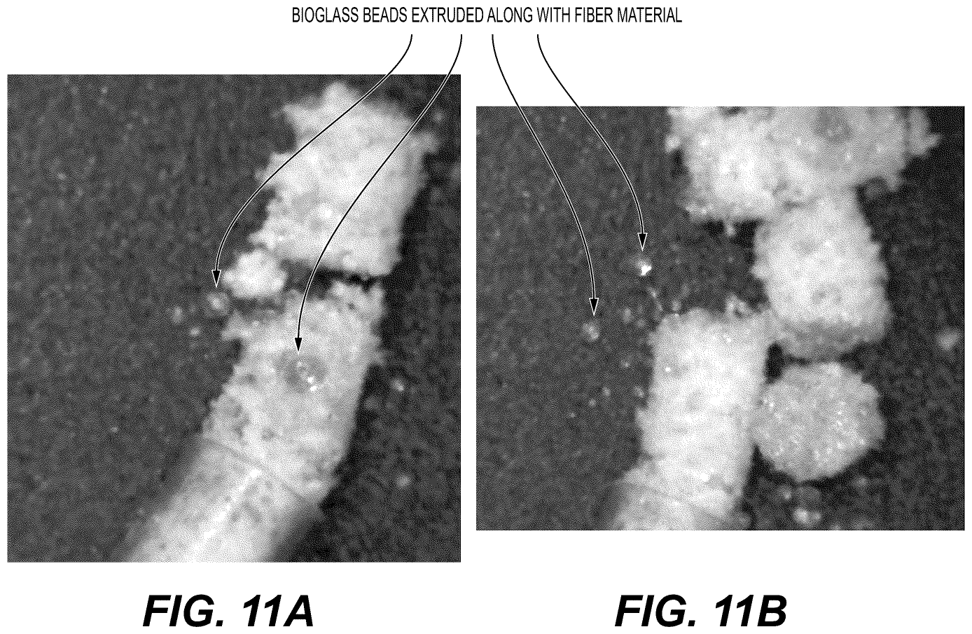

As shown in FIG. 11A and FIG. 11B, combinations of bioactive glass beads extruded along with fiber material may be illustrated wherein spherical bioactive glass beads of varying sizes may be distributed throughout a fibrous matrix. The beads may appear as light-colored, roughly spherical particles with textured surfaces, while the fiber material may form an interconnected network surrounding and interspersed among the beads. The fibers may extend in various directions, creating a three-dimensional structure that may hold the beads in place during delivery and placement procedures.

The physical arrangement of the combined bead and fiber delivery system may demonstrate how the beads may maintain their discrete spherical morphology while being integrated within the fibrous scaffold. The spherical beads may provide controlled release characteristics through their geometric properties, wherein the spherical shape may offer predictable surface area for dissolution processes. The fibrous matrix may provide structural support and enhanced surface area for ion release, creating a composite material that may combine the benefits of both bead and fiber morphologies.

The three-dimensional structure created by the fiber network may facilitate retention of the spherical beads within the composite material during handling and placement procedures. The fibers may create mechanical interlocking with the beads that may prevent separation of the components during clinical manipulation. The composite configuration may provide both the surface area benefits of the fibrous material and the controlled release characteristics of the spherical beads for periodontal applications.

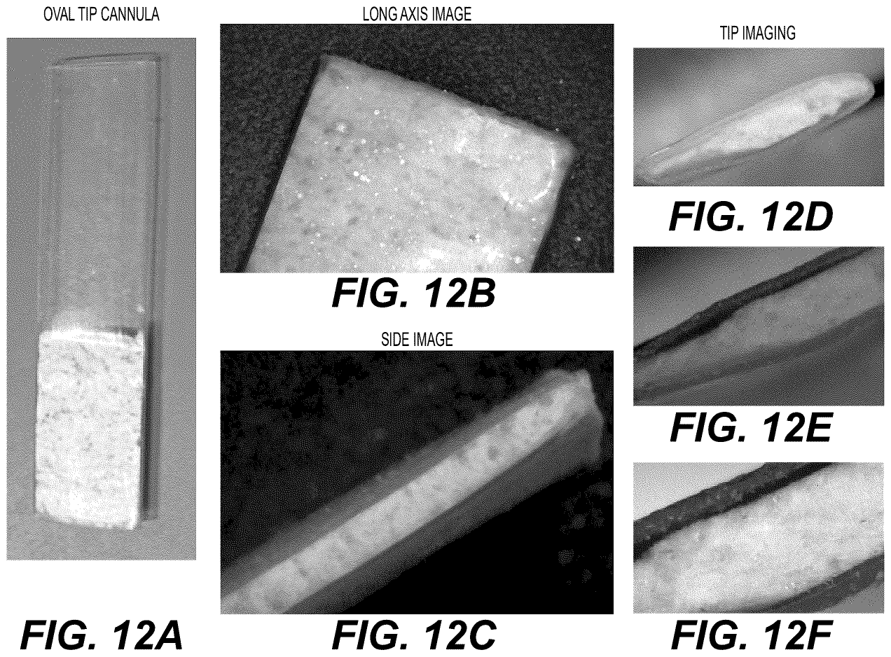

Referring to FIG. 12A, FIG. 12B, FIG. 12C, FIG. 12D, FIG. 12E, and FIG. 12F, various imaging views of an oval tip cannula delivery system are illustrated showing different perspectives and configurations of the bioactive glass fiber material within the delivery apparatus. FIG. 12A shows an oval tip cannula wherein the delivery opening exhibits an oval or rectangular cross-sectional geometry that provide increased loading density while reducing the profile needed to enter periodontal pockets. The oval tip configuration may enable greater internal volume for bioactive glass material compared to circular cross-sections of equivalent maximum dimension.

The oval tip cannula may be manufactured through heat shrinking processes wherein fluorinated ethylene propylene (FEP) tubing with a wall thickness of about 0.008 inches may be applied onto a 1.9 mm shim to create the desired oval geometry. The heat shrinking process may involve controlled heating of the FEP material to cause dimensional reduction and conforming to the shim profile. The resulting oval cross-section may optimize the balance between material loading capacity and insertion profile for periodontal applications.

A 1 mm Teflon shim may be used as an ejector pin during the manufacturing process to maintain the internal lumen dimensions while the FEP material may be heat shrunk around the forming shim. The Teflon shim may provide a non-stick surface that may facilitate removal after the heat shrinking process may be completed. The dimensional relationship between the forming shim and the ejector pin may determine the final internal geometry of the oval tip cannula.

FIG. 12B shows a long axis image of the bioactive glass fiber material wherein the compressed or aggregated mass of material is visible along its longitudinal extent. The long axis view depicts the internal structure and organization of the bioactive glass fibers within the delivery system. The image demonstrates how the fiber material may be arranged within the cannula volume to enable controlled advancement during delivery procedures.

FIG. 12C shows a side image of the bioactive glass fiber material wherein the profile and surface characteristics may be visible along the length of the material. The side view illustrates the dimensional characteristics of the compressed fiber material and shows how the material interfaces with the cannula walls. The side imaging perspective provides information about the material density and structural uniformity along the delivery axis.

FIG. 12D, FIG. 12E, and FIG. 12F show tip imaging views wherein close-up perspectives of the distal end of the delivery system may reveal the bioactive glass fiber material in its delivery configuration. The tip images display elongated fiber structures appearing as light-colored cylindrical forms with textured surfaces and visible internal structure. The tip imaging demonstrate the physical characteristics of the bioactive glass material at the delivery region wherein the fibers are positioned for extrusion into periodontal sites. The multiple tip views provide different perspectives of the material configuration and show how the bioactive glass fibers may be organized within the oval tip cannula for controlled dispensing during clinical procedures.

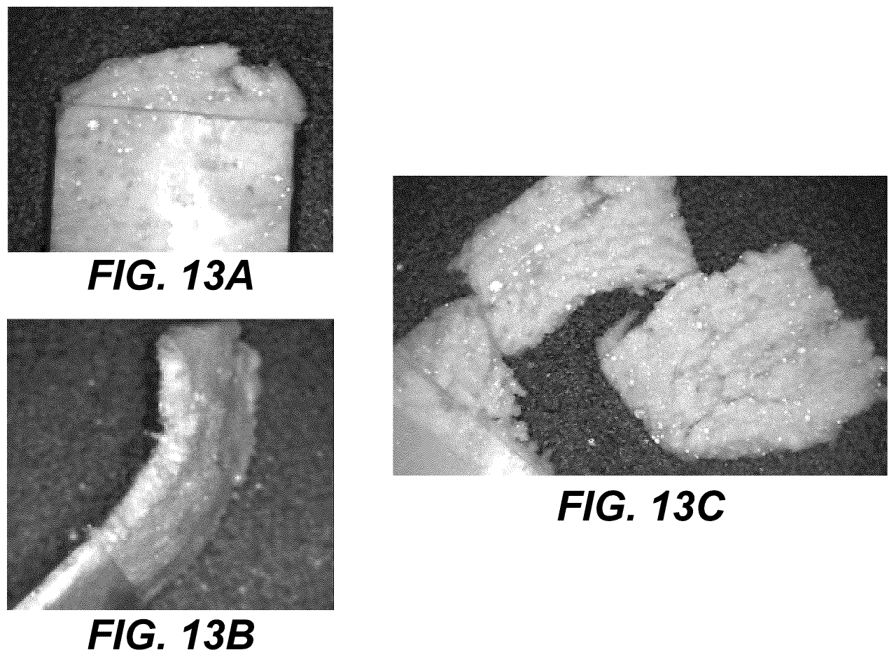

FIG. 13A, FIG. 13B, and FIG. 13C, illustrate an oval tip cannula extruding a fiber and bead mix composition wherein the delivery system enables controlled dispensing of combined bioactive glass materials. The oval tip cannula configuration may provide enhanced loading capacity while maintaining a reduced insertion profile for periodontal applications. The fiber and bead mix may combine the surface area advantages of fibrous materials with the controlled release characteristics of spherical particles to optimize therapeutic effectiveness.

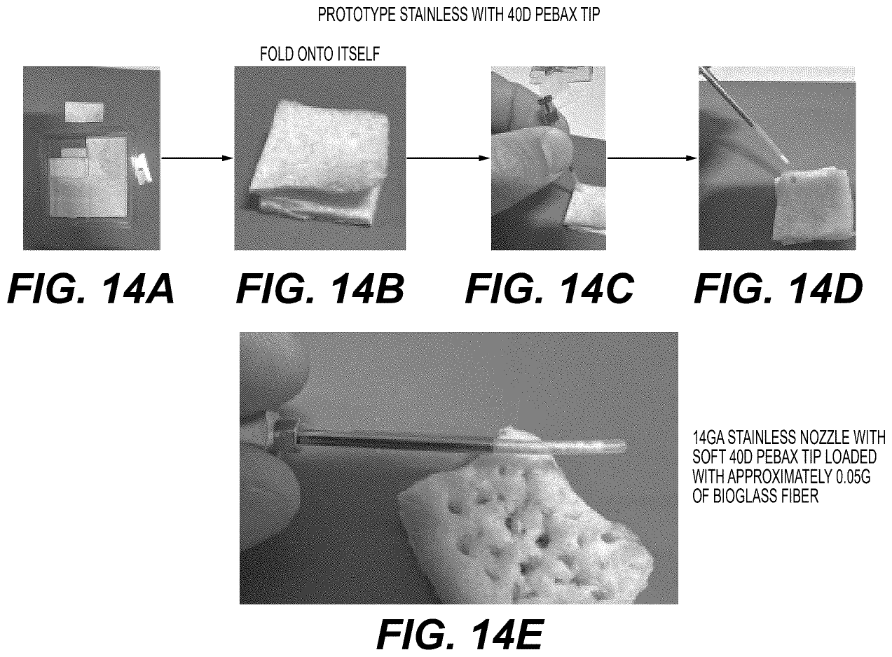

Referring to FIG. 14A, FIG. 14B, FIG. 14C, FIG. 14D, and FIG. 14E, a loading sequence s demonstrated showing how flat bioactive glass substrates may be transformed into compact delivery forms through controlled folding and loading techniques. FIG. 14A depicts a flat bioglass fiber positioned on a surface. The flat configuration may represent the initial form of the bioactive glass material before folding and loading processing.

FIG. 14B depicts the flat bioglass fiber of FIG. 14A in a folded configuration. The material demonstrates flexibility as the substrate transitions from a flat to a folded state, indicating that the bioactive glass composition may possess sufficient flexibility for folding operations without structural failure. The folded configuration may represents an intermediate stage in the transformation process wherein the material may be prepared for loading into delivery systems.

FIG. 14C depicts the folded bioglass fiber of FIG. 14B being loaded into a 14GA stainless steel nozzle with a soft 40D PEBAX tip. The loading process may involve positioning the folded bioactive glass material within the delivery system wherein the 14GA stainless steel nozzle may provide structural support while the soft 40D PEBAX tip may enable atraumatic insertion into periodontal sites.

FIG. 14D depicts the folded bioglass fiber of FIG. 14B in a partially loaded 14GA stainless steel nozzle with a soft 40D PEBAX tip, wherein the material may be positioned within the delivery system during the loading progression.

FIG. 14E depicts the folded bioglass fiber of FIG. 14B in a fully loaded 14GA stainless steel nozzle with a soft 40D PEBAX tip. The loading technique may be used to create a compact delivery form from a flat bioactive glass material that may be suitable for insertion into periodontal pockets or other dental applications. The loaded configuration may provide increased material density while maintaining the therapeutic properties of the bioactive glass composition within the delivery system.

In some embodiments, phase-invertible gel compositions may provide advanced delivery systems for periodontal therapy wherein bioactive glass particles may be suspended within thermosensitive carrier matrices that may undergo controlled phase transitions at physiological temperatures. A phase-invertible gel composition for periodontal therapy may comprise bioactive glass particles including boron oxide and calcium oxide as primary network formers combined with a phase-invertible carrier such Poloxamer 407 in water. In some embodiments, poloxamer 407 may be at a concentration of 30 percent by weight. The composition may transition from liquid to gel at body temperature, providing controlled delivery characteristics that may enhance therapeutic effectiveness.

The bioactive glass particles may be enhanced with trace elements at concentrations ranging from about 0.01% to about 20% by weight, wherein the trace elements may be selected from copper oxide, zinc oxide, strontium oxide, iron oxide, potassium oxide, magnesium oxide, sodium oxide, and phosphorus pentoxide. The trace element enhancement may provide specific biological functions including antimicrobial activity, osteoblast stimulation, and bone mineralization promotion. The concentration range may be optimized to achieve desired therapeutic effects while maintaining compatibility with the phase-invertible carrier system.

Poloxamer 407 may function as a thermosensitive polymer that may exhibit reverse thermal gelation properties wherein aqueous solutions may transition from liquid states at low temperatures to gel states at elevated temperatures. The phase transition temperature may be controlled through polymer concentration and solution composition to achieve gelation at body temperature conditions (e.g., about 98° F.). The 30 percent by weight concentration of Poloxamer 407 in water may provide optimal phase transition characteristics for periodontal applications wherein the composition may remain liquid during preparation and delivery procedures while forming stable gels upon contact with body temperature environments.

The phase transition from liquid to gel at body temperature may provide controlled delivery characteristics wherein the composition may be easily injected as a liquid through syringe systems while forming stable gel matrices upon placement in periodontal sites. The liquid state during delivery may enable the composition to flow into irregular defect geometries and confined periodontal spaces that may be inaccessible to solid delivery forms. The gel formation at body temperature may provide sustained retention of the bioactive glass particles at treatment sites through immobilization within the gel matrix.

In some embodiments, the Poloxamer 407 concentration may be adjusted within a range of about 15% to about 60% by weight to achieve varying phase transition temperatures and gel strength characteristics. The concentration may be about 15%, about 20%, about 25%, about 30%, about 35%, about 40%, about 45%, about 50%, about 55%, or about 60% by weight. Lower concentrations such as about 15% to about 25% by weight may provide phase transition temperatures above body temperature, wherein the composition may remain in liquid form during initial placement and may undergo delayed gelation as the temperature equilibrates with surrounding tissues. Intermediate concentrations such as about 30% to about 40% by weight may provide phase transition temperatures at or near body temperature, enabling rapid gel formation upon contact with periodontal tissues. Higher concentrations such as about 45% to about 60% by weight may provide phase transition temperatures below body temperature, wherein the composition may form gels at room temperature or may require cooling during preparation and delivery procedures.

The preparation process for the phase-invertible gel composition may involve compounding the Poloxamer 407 solution at temperatures below 40 degrees Fahrenheit to form a viscous liquid that may maintain flowable characteristics during preparation procedures. The low temperature preparation may prevent premature gelation during mixing operations while ensuring complete dissolution of the Poloxamer 407 polymer. The viscous liquid state achieved at low temperatures may facilitate uniform distribution of bioactive glass particles throughout the carrier matrix.

The phase transition from liquid to gel at body temperature may provide controlled delivery characteristics wherein the composition may be easily injected as a liquid through syringe systems while forming stable gel matrices upon placement in periodontal sites. The liquid state during delivery may enable the composition to flow into irregular defect geometries and confined periodontal spaces that may be inaccessible to solid delivery forms. The gel formation at body temperature may provide sustained retention of the bioactive glass particles at treatment sites through immobilization within the gel matrix.

The preparation process for the phase-invertible gel composition may involve compounding the Poloxamer 407 solution at temperatures below 40 degrees Fahrenheit to form a viscous liquid that may maintain flowable characteristics during preparation procedures. The low temperature preparation may prevent premature gelation during mixing operations while ensuring complete dissolution of the Poloxamer 407 polymer. The viscous liquid state achieved at low temperatures may facilitate uniform distribution of bioactive glass particles throughout the carrier matrix.

The bioactive glass particles may be added to the Poloxamer solution at concentrations of about 10 percent to about 70 percent by weight to achieve desired therapeutic loading while maintaining appropriate flow characteristics for syringe delivery. The concentration range may be selected based on specific clinical requirements wherein lower concentrations may provide enhanced flow properties while higher concentrations may deliver greater quantities of bioactive material per unit volume. The particle loading may be optimized to balance therapeutic effectiveness with delivery performance characteristics.

The mixing process may involve thorough blending of the bioactive glass particles with the cooled Poloxamer 407 solution to achieve uniform particle distribution throughout the carrier matrix. The low temperature conditions during mixing may maintain the liquid state of the carrier while preventing particle settling or aggregation. The uniform distribution may ensure consistent therapeutic ion release patterns throughout the gel matrix after phase transition occurs at body temperature.

The composition may be transferred to a syringe while maintaining cool storage conditions until clinical use to preserve the liquid state and prevent premature gelation. The syringe loading may be performed under controlled temperature conditions wherein the composition may remain flowable for accurate volumetric dispensing. The cool storage conditions may be maintained through refrigeration or other temperature control methods that may keep the composition below the phase transition temperature until clinical application.

The phase-invertible gel composition may comprise Type I collagen at concentrations ranging from about 0.1% to about 30% by weight, wherein the collagen concentration may be from about 0.1% to about 5% by weight in certain embodiments. In other embodiments, Type I collagen may be present at a concentration of about 0.5% to about 1.5% by weight. In other embodiments, Type I collagen may be present at a concentration of about 2% to 5%. The composition may further include approximately 60% by weight of bioactive glass particles comprising boron oxide and calcium oxide as primary network formers. The remaining portion of the composition may include water and Poloxamer 407, which together may provide the thermosensitive carrier matrix that may enable the phase transition from liquid to gel at body temperature conditions.

The syringe storage system may enable convenient clinical use wherein the phase-invertible gel composition may be maintained in ready-to-use configurations while preserving the liquid state through temperature control. The syringe packaging may provide sterile storage conditions while enabling direct delivery without transfer procedures that may introduce contamination or temperature variations. The cool storage requirements may be compatible with standard clinical refrigeration systems commonly available in dental practice environments.

Upon delivery into periodontal sites, the composition may undergo phase transition from liquid to gel state as the temperature increases to body temperature levels. The phase transition may occur rapidly upon contact with periodontal tissues, creating a stable gel matrix that may immobilize the bioactive glass particles within the treatment site. The gel formation may provide sustained retention of bioactive glass particles at treatment sites through formation of a stable gel matrix that may resist displacement by oral fluids and mechanical forces.

The stable gel matrix may ensure that therapeutic concentrations of bioactive ions may be maintained in the treatment area for sufficient duration to achieve desired biological responses. The gel structure may provide controlled release characteristics wherein the bioactive glass particles may undergo dissolution within the gel matrix while the released ions may diffuse through the gel structure to reach surrounding periodontal tissues. The sustained retention may eliminate the need for frequent reapplication and may improve patient compliance with treatment protocols.

The composition may dissolve in interstitial fluids to release bioactive ions that may stimulate hydroxyapatite formation and create an alkaline environment within periodontal treatment sites. The dissolution process may occur gradually as interstitial fluids may penetrate the gel matrix and contact the embedded bioactive glass particles. The ion release may provide calcium, phosphate, and other therapeutic elements that may serve as building blocks for new bone matrix formation while creating chemical conditions that may inhibit bacterial growth.

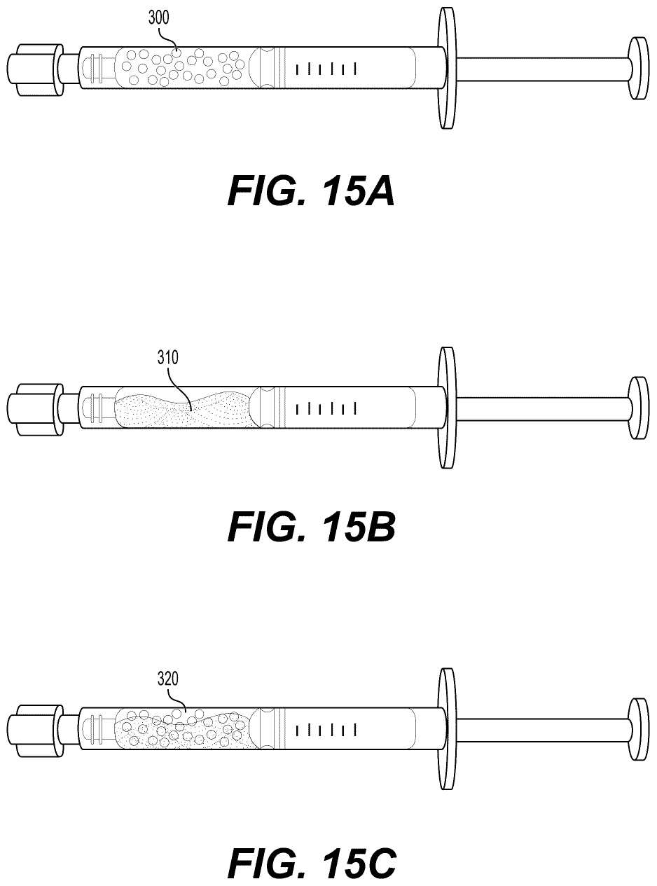

Referring to FIG. 15A, FIG. 15B, and FIG. 15C, show the preparation process of an embodiment of the phase-invertible gel compositions. FIG. 15A depicts a syringe filled with bioactive glass beads 300 wherein the beads may be maintained in dry form prior to mixing with carrier solutions. The bioactive glass beads 300 may comprise spherical particles with diameters ranging from 150 to 300 microns that may include boron oxide and calcium oxide as primary network formers along with trace elements at concentrations of about 0.01% to about 20% by weight. The syringe configuration may enable controlled dispensing of predetermined quantities of bioactive glass beads 300 for mixing procedures.

FIG. 15B may illustrate a syringe filled with phase-invertible carrier 310 such as Poloxamer 407 in liquid form wherein the thermosensitive polymer may be maintained at temperatures below 40 degrees Fahrenheit to preserve flowable characteristics. The phase-invertible carrier 310 may be prepared at concentrations ranging from about 15% to about 60% by weight in water to achieve desired phase transition temperatures and gel strength properties. The liquid state of the phase-invertible carrier 310 may be maintained through temperature control during storage and handling procedures to prevent premature gelation prior to mixing with bioactive glass beads 300.

FIG. 15C shows the formation of a phase invertible gel composition 320 that includes the bioactive glass beads 300 from FIG. 15A suspended in the phase invertible carrier 310 from FIG. 15B. The phase invertible gel composition 320 may include bioactive glass beads 300 uniformly distributed throughout the phase-invertible carrier 310 wherein the mixing process may ensure consistent particle suspension throughout the composition volume. The bioactive glass beads 300 may be present at concentrations of 10 to 70 percent by weight within the phase invertible gel composition 320 to achieve desired therapeutic loading while maintaining appropriate flow characteristics for syringe delivery. The phase invertible gel composition 320 may maintain liquid state characteristics at cool storage temperatures while retaining the capability to undergo phase transition to gel state upon exposure to body temperature conditions.

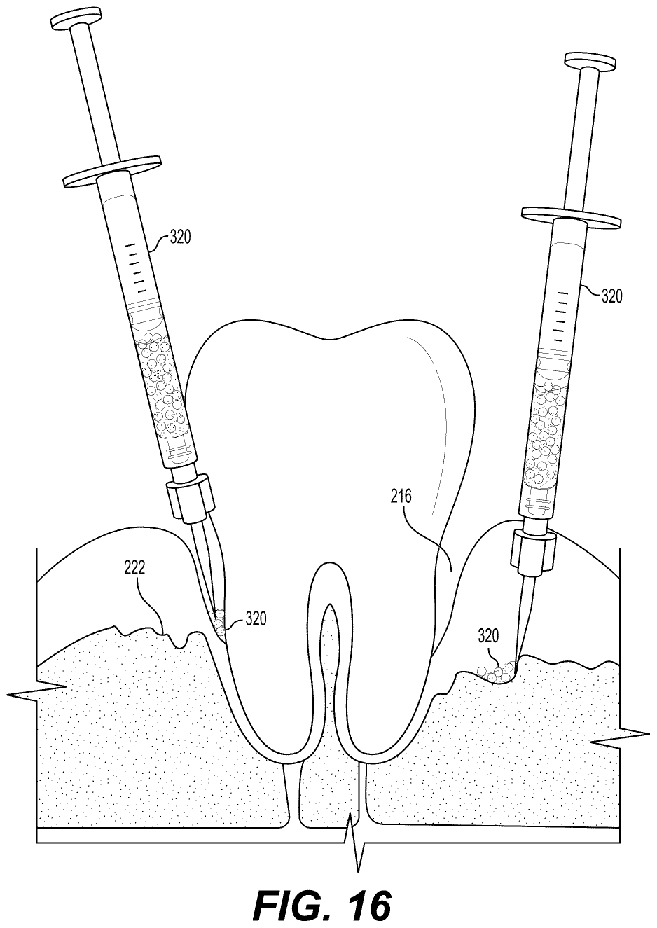

Referring to FIG. 16, the clinical application of the phase invertible gel composition 320 may is illustrated wherein the phase-invertible gel composition may be delivered to multiple periodontal treatment sites through syringe-based injection techniques. The phase invertible gel composition 320 may be delivered via syringe A to periodontal pocket 216 wherein the composition may flow into the confined space between the tooth and surrounding periodontal tissues. The liquid state of the phase invertible gel composition 320 during delivery may enable the composition to conform to the irregular geometry of the periodontal pocket 216 while ensuring complete filling of the defect space. Upon contact with body temperature conditions within the periodontal pocket 216, the phase invertible gel composition 320 may undergo phase transition from liquid to gel state, creating a stable matrix that may immobilize the bioactive glass beads 300 within the treatment site. The phase invertible gel composition 320 may also be delivered via syringe B to areas of bone loss 222 wherein the composition may fill voids created by periodontal disease processes. The delivery to areas of bone loss 222 may provide bioactive glass beads 300 in direct contact with remaining periodontal bone structures wherein the dissolution of the beads may release therapeutic ions that may stimulate bone regeneration processes. The gel formation at body temperature may ensure sustained retention of the bioactive glass beads 300 within the areas of bone loss 222 while enabling controlled ion release over extended treatment periods. The dual delivery approach illustrated in FIG. 16 may demonstrate the versatility of the phase-invertible gel composition for treating multiple types of periodontal defects wherein the flowable liquid characteristics may enable access to confined spaces while the gel formation may provide long-term retention at treatment sites.

The phase-invertible carrier 310 may undergo gradual degradation and clearance from the treatment site over time while the bioactive glass beads 300 may remain to provide sustained therapeutic benefits. The phase-invertible carrier 310 may dissolve and be cleared through natural physiological processes wherein the polymer may be eliminated from the periodontal site through diffusion into surrounding tissues and subsequent systemic clearance. The carrier degradation may occur over a period of days to weeks depending on the concentration and molecular weight of the Poloxamer 407 used in the formulation. As the carrier matrix may dissolve and clear from the treatment site, the bioactive glass beads 300 may remain in position within the periodontal defect wherein the beads may continue to undergo controlled dissolution and release therapeutic ions. The persistence of the bioactive glass beads 300 after carrier clearance may enable extended therapeutic effects that may continue for weeks to months following initial delivery. The differential clearance rates between the carrier and the bioactive glass beads 300 may provide a two-phase therapeutic approach wherein the initial gel matrix may provide immediate stabilization and retention while the subsequent presence of the beads alone may deliver long-term regenerative benefits through sustained ion release.

The alkaline environment created through bioactive glass dissolution may discourage bacterial colonization by creating pH conditions that may be unfavorable for periodontal pathogens. The alkaline conditions may be particularly effective against neutrophilic bacteria such as Porphyromonas gingivalis and Treponema denticola that may demonstrate reduced viability outside neutral pH ranges. The antimicrobial effects may complement the regenerative properties of the bioactive glass particles to provide comprehensive periodontal therapy.

The hydroxyapatite formation stimulated by the released bioactive ions may promote bone regeneration processes within periodontal defects through the provision of mineral components that may serve as nucleation sites for new bone formation. The calcium and phosphate ions released from the bioactive glass particles may combine with endogenous biological components to form hydroxyapatite crystals that may integrate with existing bone structures. The mineralization process may be enhanced by the controlled ion concentrations maintained through the sustained release characteristics of the gel matrix system.

The phase-invertible gel composition may provide advantages over conventional delivery systems through the combination of easy injection characteristics during placement procedures with sustained retention properties after gel formation. The dual-phase behavior may enable clinicians to deliver precise quantities of bioactive glass material into periodontal sites while ensuring long-term retention for sustained therapeutic effects. The gel matrix may protect the bioactive glass particles from premature dissolution while enabling controlled release over extended treatment periods.

In some embodiments, syringe-delivered microsphere compositions may be suitable for accessing confined or irregular periodontal spaces that may be difficult to treat with solid delivery forms. The flowable characteristics of the suspension may enable the material to conform to complex defect geometries while ensuring complete filling of treatment sites. The microsphere delivery system may provide controlled therapeutic ion release through the dissolution of individual spherical particles within the carrier matrix, enabling sustained treatment effects over extended periods.

Syringe-delivered microsphere compositions may comprise spherical bioactive glass particles ranging in diameter from 150 to 300 microns that may be suspended in carrier solutions for minimally invasive delivery applications. The microsphere size range may be selected to enable delivery through standard syringe and cannula systems while providing appropriate dissolution characteristics for periodontal applications. The spherical geometry may provide predictable flow properties through delivery systems while optimizing surface area for ion release processes.

The carrier solutions may include methylcellulose-based suspensions that may be prepared with a 10 percent by weight mixture of hydroxypropylmethyl cellulose with water. The hydroxypropylmethyl cellulose may provide appropriate viscosity characteristics for syringe delivery while maintaining suspension properties that may prevent settling of the bioactive glass particles. The 10 percent concentration may be selected to achieve optimal flow characteristics while providing sufficient viscosity to maintain particle suspension during storage and delivery procedures. The bioactive glass particles may be added to the hydroxypropylmethyl cellulose solution at concentrations of about 10 to about 50 percent by weight to form a suspension suitable for syringe delivery. The concentration range may be adjusted based on specific clinical requirements wherein lower concentrations may provide enhanced flow characteristics while higher concentrations may deliver greater quantities of therapeutic material per unit volume. The suspension may maintain uniform distribution of bioactive glass particles throughout the carrier solution.

The mixture may be drawn into a syringe of suitable size with a needle or cannula of suitable gauge applied for delivery into periodontal treatment sites. The syringe system may enable controlled dispensing of the microsphere suspension into periodontal defects, small or irregular periodontal voids, and for implant packing applications. The delivery method may provide minimally invasive application techniques that may reduce patient discomfort while enabling precise placement of therapeutic materials.

In some embodiments, chewing gum formulations may provide an alternative delivery mechanism for bioactive glass particles wherein the particles may be infused within gum base materials to enable gradual release of therapeutic ions through stimulated salivary flow. The chewing gum delivery system may incorporate bioactive glass particles comprising boron oxide and calcium oxide as primary network formers along with trace elements at concentrations ranging from about 0.01% to about 20% by weight. The mechanical action of chewing may facilitate the release of bioactive glass particles from the gum matrix while simultaneously stimulating salivary production that may enhance ion distribution throughout the oral cavity.

The bioactive glass particles within the chewing gum formulation may undergo controlled release during the chewing process wherein the mechanical forces applied during mastication may gradually expose embedded particles to salivary fluids. The particle release rate may be controlled through the selection of gum base materials and particle loading concentrations to achieve sustained therapeutic ion delivery over extended chewing periods. The stimulated salivary flow resulting from the chewing action may facilitate distribution of released bioactive ions to periodontal tissues throughout the oral cavity.

The chewing gum delivery mechanism may be particularly suitable for preventative care applications wherein regular use may maintain therapeutic ion concentrations in oral fluids to discourage bacterial colonization and support periodontal health. The convenience of the chewing gum format may encourage patient compliance with preventative treatment regimens while providing sustained therapeutic benefits through routine use. The gradual ion release characteristics may provide long-term exposure to therapeutic concentrations without requiring clinical intervention procedures.

The chewing gum formulation may be optimized for early-stage periodontal disease management wherein the sustained ion release may address initial bacterial colonization and inflammatory processes before advanced periodontal breakdown occurs. The alkaline environment created by dissolving bioactive glass particles may neutralize acidic conditions that may contribute to periodontal disease progression while providing antimicrobial effects against pathogenic bacteria. The mechanical stimulation of periodontal tissues during chewing may complement the chemical therapeutic effects by promoting circulation and tissue health.

In other embodiments, dissolvable strip configurations may provide targeted delivery mechanisms wherein thin strips of bioactive glass material may be adhered to the gumline and may slowly dissolve to release antimicrobial ions for localized therapy applications. The dissolvable strips may be manufactured from bioactive glass compositions that may be formed into thin, flexible sheets through compression molding or casting processes. The strip geometry may enable precise placement along specific gumline regions wherein localized periodontal treatment may be desired.

The adhesion characteristics of the dissolvable strips may be achieved through the incorporation of biocompatible adhesive materials or through surface modifications that may promote adherence to moist oral tissues. The strips may maintain contact with gumline tissues for predetermined durations while undergoing controlled dissolution processes that may release therapeutic ions directly to targeted periodontal sites. The localized delivery approach may provide concentrated therapeutic effects while minimizing systemic exposure to bioactive materials.

The dissolution rate of the strips may be controlled through material composition and thickness parameters to achieve desired treatment durations ranging from minutes to hours depending on specific therapeutic requirements. The slow dissolution process may provide sustained release of antimicrobial ions that may create localized alkaline conditions unfavorable for bacterial growth while stimulating tissue healing responses. The strip format may enable treatment of specific periodontal sites without affecting adjacent healthy tissues.

The dissolvable strips may be particularly advantageous for treating localized periodontal defects wherein targeted therapy may be more appropriate than broad-spectrum treatments. The strips may be sized and shaped to match specific defect geometries while providing controlled therapeutic ion release directly to affected tissues. The adherent properties may ensure that therapeutic materials remain in contact with treatment sites for sufficient duration to achieve desired biological responses.

In some embodiments, mouth spray and rinse formulations may provide broad-area delivery mechanisms wherein bioactive glass nanoparticles or dissolved ions may be dispersed in liquid carriers for quick, non-invasive application over extensive oral surfaces. The spray delivery system may utilize atomization techniques to create fine droplets containing bioactive glass nanoparticles that may be distributed throughout the oral cavity through controlled spray patterns. The nanoparticle size may be optimized to remain suspended in the liquid carrier while providing enhanced surface area for rapid dissolution and ion release.

The liquid formulations may contain bioactive glass nanoparticles at concentrations that may provide therapeutic ion levels while maintaining appropriate flow characteristics for spray or rinse applications. The nanoparticles may undergo rapid dissolution in oral fluids to release calcium, phosphate, and other therapeutic ions that may enhance oral pH conditions and reduce bacterial loads throughout the oral cavity. The broad distribution achieved through spray or rinse applications may provide comprehensive coverage of periodontal tissues that may be difficult to access through localized delivery methods.

The mouth spray formulations may be particularly suitable for maintenance therapy applications wherein regular use may maintain favorable oral conditions for periodontal health while providing antimicrobial effects against pathogenic bacteria. The convenience of spray application may enable frequent use throughout the day to maintain therapeutic ion concentrations in oral fluids. The rapid application and broad coverage characteristics may make spray formulations suitable for patients with extensive periodontal involvement requiring comprehensive treatment approaches.

The rinse formulations may provide extended contact times compared to spray applications wherein the liquid may be retained in the oral cavity for predetermined durations to maximize therapeutic ion exposure. The rinse format may enable controlled swishing actions that may distribute the bioactive glass nanoparticles to all oral surfaces while providing mechanical cleansing effects that may complement the chemical therapeutic benefits. The extended contact time may enhance the dissolution of nanoparticles and the subsequent release of therapeutic ions to periodontal tissues.

The enhancement of oral pH through bioactive glass dissolution may create alkaline conditions that may discourage the growth of acidogenic bacteria responsible for periodontal disease progression. The pH modification may neutralize acidic metabolic products produced by pathogenic bacteria while creating environmental conditions that may favor beneficial oral microorganisms. The broad-area pH enhancement achieved through spray or rinse applications may provide comprehensive antimicrobial effects throughout the oral cavity.

Collagen matrix delivery systems may incorporate bioactive glass compositions within collagen frameworks to improve osteogenic performance and provide supportive microenvironments for bone regeneration applications. The collagen matrices may be formed from Type I collagen that may provide structural scaffolding for cellular attachment and tissue integration while serving as a carrier for bioactive glass particles. The combination of collagen structural support with bioactive glass ion release may create synergistic effects that may enhance bone formation processes beyond the capabilities of either component alone.

The bioactive glass compositions may be combined with collagen at concentrations of about 1% to about 50% by weight for soft-tissue or gum applications wherein the collagen component may provide biocompatible scaffolding that may support cellular infiltration and tissue regeneration. The concentration range may be selected based on specific application requirements wherein lower collagen concentrations may be used when enhanced bioactive glass effects may be desired while higher collagen concentrations may be employed when structural support and cellular guidance may be priorities.

The fibrous and bead compositions of bioactive glass may be embedded within collagen frameworks through various manufacturing processes including solution casting, freeze-drying, or compression molding techniques. The embedding process may ensure uniform distribution of bioactive glass components throughout the collagen matrix while maintaining the structural integrity of both materials. The embedded bioactive glass particles may undergo controlled dissolution within the collagen matrix while the collagen may provide a degradable scaffold that may be replaced by natural tissue during healing processes.

The collagen matrix may provide a supportive microenvironment for bone regeneration through the presentation of cellular attachment sites and the release of collagen degradation products that may stimulate cellular activities. The Type I collagen may be recognized by cellular receptors that may promote osteoblast attachment, proliferation, and differentiation processes. The gradual degradation of the collagen matrix may create space for new tissue formation while providing amino acids and peptides that may support cellular metabolism and tissue synthesis.

The combination of collagen scaffolding with bioactive glass ion release may create favorable conditions for bone regeneration wherein the collagen may provide immediate structural support while the bioactive glass may supply the mineral components needed for hydroxyapatite formation. The temporal coordination of collagen degradation with bioactive glass dissolution may ensure that structural support may be maintained during the initial phases of tissue regeneration while mineral supplementation may continue throughout the bone formation process.

The collagen matrix delivery system may be particularly suitable for applications wherein both soft tissue and hard tissue regeneration may be desired, such as in periodontal defects involving both gingival recession and alveolar bone loss. The collagen component may support gingival tissue regeneration while the bioactive glass component may promote alveolar bone formation, providing comprehensive tissue regeneration capabilities within a single delivery system.

Auger delivery systems may provide mechanical delivery mechanisms wherein bioactive glass materials may be delivered through screw drive mechanisms that may enable controlled advancement of materials through delivery cannulas. The auger system may comprise a helical screw mechanism positioned within a delivery cannula wherein rotation of the screw may advance bioactive glass material from a loading chamber toward the distal delivery opening. The screw drive mechanism may provide precise control over material delivery rates through the regulation of rotational speed and direction.

The auger delivery mechanism may be particularly suitable for delivering bioactive glass materials with varying consistency characteristics including powders, granules, fibers, or paste formulations. The helical screw configuration may provide positive displacement characteristics that may ensure consistent material advancement regardless of material flow properties. The mechanical advantage provided by the screw mechanism may enable controlled delivery of materials that may be difficult to dispense through pressure-based systems.

The screw drive mechanism may enable bidirectional material movement wherein forward rotation may advance material toward the delivery opening while reverse rotation may retract material back into the loading chamber. The bidirectional capability may provide enhanced control over material placement by enabling precise positioning adjustments during delivery procedures. The mechanical control may eliminate the variability associated with manual pressure application while providing reproducible delivery characteristics.

The auger system may incorporate loading chambers with sufficient capacity to contain predetermined quantities of bioactive glass material for complete treatment procedures. The loading chamber may be designed to accommodate various material forms while ensuring consistent feeding to the screw mechanism. The chamber configuration may prevent material bridging or jamming that may interfere with smooth operation of the auger system.

Magazine loading systems may provide delivery mechanisms wherein solid bioactive glass materials may be loaded into delivery systems through ejector pin apparatus similar to magazine mechanisms used in mechanical devices. The magazine system may comprise a loading chamber that may contain multiple units of solid bioactive glass materials arranged in sequential order for individual delivery. The ejector pin apparatus may advance individual bioactive glass units from the magazine chamber toward the delivery opening through controlled mechanical advancement.

The magazine loading mechanism may be particularly suitable for delivering preformed bioactive glass units such as wedges, pellets, rods, or other shaped configurations that may require individual placement procedures. The sequential loading capability may enable multiple treatments to be performed using a single delivery device while ensuring consistent material characteristics for each delivered unit. The magazine capacity may be designed to accommodate sufficient bioactive glass units for complete treatment protocols.

The ejector pin apparatus may provide controlled advancement of individual bioactive glass units through spring-loaded mechanisms, manual advancement systems, or motorized drive mechanisms depending on specific application requirements. The ejector pin may be sized to provide appropriate contact with bioactive glass units while enabling smooth advancement through the delivery system. The pin mechanism may incorporate features to prevent jamming or misalignment during operation.

The magazine system may incorporate indexing mechanisms that may position successive bioactive glass units for delivery while preventing multiple units from being advanced simultaneously. The indexing system may ensure that individual units may be delivered in controlled sequences while maintaining proper alignment within the delivery system. The sequential delivery capability may enable precise placement of multiple bioactive glass units within complex periodontal defects requiring staged treatment approaches.

The magazine loading system may provide advantages for clinical applications wherein multiple periodontal sites may require treatment during single appointment procedures. The preloaded magazine may eliminate the need for individual loading procedures between treatment sites while ensuring consistent material characteristics for each delivered unit. The system may reduce treatment time and improve procedural efficiency while maintaining precise control over material placement at each treatment site.

A method of stabilizing loose teeth may comprise accessing a periodontal void adjacent to a loose tooth through clinical procedures that may prepare the treatment site for bioactive glass delivery. The method may begin with assessment of periodontal conditions to identify areas wherein bone loss may have created voids that may compromise tooth stability. The periodontal void may represent spaces between the tooth root and surrounding periodontal bone wherein disease processes may have resulted in tissue destruction and structural compromise.

Accessing the periodontal void may comprise scaling and root planing to prepare the periodontal pocket and remove bacterial deposits that may interfere with healing processes and bioactive glass effectiveness. The scaling procedure may involve mechanical removal of calculus deposits from tooth surfaces using ultrasonic or hand instruments that may eliminate bacterial biofilms and mineralized deposits. The root planing process may smooth root surfaces to remove bacterial toxins and create favorable conditions for tissue reattachment and healing.

The scaling and root planing procedures may create clean periodontal environments wherein bacterial loads may be reduced to levels that may not interfere with regenerative processes. The removal of bacterial deposits may eliminate sources of ongoing inflammation and tissue destruction while creating conditions that may be favorable for bioactive glass dissolution and ion release. The prepared periodontal pocket may provide access to the periodontal void while ensuring that delivered bioactive glass materials may contact clean tissue surfaces.

The method may continue with delivering a bioactive glass composition into the periodontal void using a delivery system selected from multiple options that may be chosen based on specific clinical requirements and defect characteristics. The bioactive glass composition may comprise boron oxide and calcium oxide as primary network formers that may provide the structural foundation for controlled dissolution and ion release processes. The composition may be enhanced with trace elements at concentrations of about 0.01% to about 20% by weight to provide specific therapeutic functions including antimicrobial activity and bone formation stimulation.

The delivery system may comprise compacted wedges shaped to fit into periodontal voids wherein the wedge geometry may enable precise placement within the prepared periodontal pocket. The compacted wedges may be dimensioned to match the specific geometry of the periodontal void while providing mechanical stability during placement procedures. The wedge shape may facilitate insertion into narrow periodontal spaces while maximizing contact with surrounding tissue surfaces to optimize therapeutic ion delivery.

The delivery system may alternatively comprise syringe-delivered microspheres suspended in carrier solutions that may enable minimally invasive delivery into periodontal voids through controlled injection techniques. The microspheres may range in diameter from 150 to 300 microns to ensure compatibility with syringe delivery systems while providing appropriate dissolution characteristics. The carrier solutions may maintain microsphere suspension while providing flowable properties that may enable delivery into irregular or confined periodontal spaces.

The delivery system may further comprise fibrous and bead compositions that may combine the surface area advantages of fibrous materials with the controlled release characteristics of spherical particles. The fibrous components may provide enhanced surface area for ion release while the bead components may offer predictable dissolution patterns through their spherical geometry. The combined composition may be delivered into periodontal voids through various placement techniques depending on the specific configuration and clinical requirements.

The bioactive glass composition may be combined with Type I collagen at concentrations of about 0.1% to about 30% by weight for bone scaffold applications wherein the collagen component may provide structural scaffolding for cellular attachment and tissue integration. The Type I collagen may serve as a framework for hydroxyapatite deposition while promoting osteoblast adhesion and cellular differentiation processes. The collagen concentration may be selected to optimize the balance between structural support and bioactive glass therapeutic effects for specific periodontal applications.

The method may conclude with allowing the bioactive glass composition to provide mechanical stabilization while releasing bioactive ions for bone regeneration within the periodontal void. The mechanical stabilization may result from the physical presence of the bioactive glass material filling the void space and providing structural support to the compromised tooth. The bioactive ion release may occur through controlled dissolution processes wherein calcium, phosphate, and other therapeutic ions may be released to stimulate osteoblast activity and bone formation.

The bioactive ions released from the composition may stimulate hydroxyapatite formation through the provision of mineral components that may serve as building blocks for new bone matrix. The ion release may create favorable chemical conditions for bone cell proliferation and differentiation while providing the raw materials needed for mineralization processes. The combination of mechanical support and biological stimulation may address both immediate stabilization needs and long-term regenerative goals in periodontal therapy.

A method of creating antimicrobial conditions in periodontal pockets may comprise delivering a phase-invertible gel composition into a periodontal pocket wherein the composition may undergo controlled phase transitions to provide sustained therapeutic effects. The phase-invertible gel composition may comprise bioactive glass particles suspended within a thermosensitive carrier matrix that may transition from liquid to gel state upon exposure to body temperature conditions. The delivery process may utilize syringe-based injection techniques that may enable precise placement of the composition within periodontal pockets.

The phase-invertible gel composition may be delivered into the periodontal pocket while in liquid state to enable flow into irregular defect geometries and confined spaces that may be inaccessible to solid delivery forms. The liquid state during delivery may facilitate complete filling of periodontal pockets while ensuring intimate contact between the composition and periodontal tissue surfaces. The injection process may be controlled to deliver appropriate volumes of composition based on pocket dimensions and therapeutic requirements.

The composition may transition from liquid to gel at body temperature through the thermosensitive properties of the Poloxamer 407 carrier system that may undergo reverse thermal gelation upon temperature elevation. The phase transition may occur rapidly upon contact with periodontal tissues wherein the composition may form a stable gel matrix that may immobilize the bioactive glass particles within the treatment site. The gel formation may provide sustained retention of therapeutic materials while preventing displacement by oral fluids and mechanical forces.

The method may continue with allowing the composition to dissolve in interstitial fluids to create an alkaline environment that may inhibit periodontal pathogens while promoting regenerative processes. The dissolution process may occur gradually as interstitial fluids may penetrate the gel matrix and contact the embedded bioactive glass particles. The controlled dissolution may release bioactive ions including calcium, phosphate, and other therapeutic elements that may modify local chemical conditions.

The alkaline environment created through bioactive glass dissolution may inhibit periodontal pathogens including Porphyromonas gingivalis and Treponema denticola that may demonstrate reduced viability outside neutral pH ranges. The elevated pH conditions may create unfavorable environments for bacterial proliferation while supporting beneficial cellular activities involved in tissue healing and regeneration. The antimicrobial effects may complement the regenerative properties of the bioactive glass particles to provide comprehensive periodontal therapy.

The method may promote bone regeneration through release of bioactive ions that may stimulate cellular processes involved in bone formation and tissue repair. The released ions may provide mineral components for hydroxyapatite formation while creating chemical signals that may enhance osteoblast activity and bone matrix synthesis. The sustained ion release characteristics of the gel matrix system may maintain therapeutic concentrations over extended periods to support ongoing regenerative processes.

The antimicrobial and regenerative effects achieved through the method may address multiple aspects of periodontal disease pathology including bacterial infection, inflammatory processes, and tissue destruction. The alkaline conditions may neutralize acidic metabolic products produced by pathogenic bacteria while the bioactive ion release may stimulate repair mechanisms that may restore periodontal tissue integrity. The comprehensive therapeutic approach may provide superior clinical outcomes compared to treatments that address only individual aspects of periodontal disease.

The method may be particularly suitable for treating periodontal pockets wherein bacterial colonization may be a primary concern while regenerative therapy may also be desired. The phase-invertible gel delivery system may enable precise placement of therapeutic materials within confined periodontal spaces while ensuring sustained retention for extended treatment effects. The dual antimicrobial and regenerative capabilities may provide comprehensive treatment approaches that may address both immediate infection control needs and long-term tissue restoration goals.

A number of implementations have been described. Nevertheless, it will be understood that various modifications may be made without departing from the spirit and scope of the disclosure. Accordingly, other implementations are within the scope of the following claims.

What is claimed is:

1. A bioactive glass delivery system for periodontal therapy, comprising:
a cannula having a proximal end and a distal end with an internal lumen extending therebetween;
bioactive glass material loaded within the internal lumen, wherein the bioactive glass fiber material comprises boron oxide and calcium oxide as primary network formers and one or more trace elements selected from copper oxide, zinc oxide, strontium oxide, iron oxide, potassium oxide, magnesium oxide, sodium oxide, and phosphorus pentoxide; and
an obturator positioned within the internal lumen and configured to advance the bioactive glass fiber material through the distal end of the cannula.

2. The bioactive glass delivery system of claim 1, wherein the cannula comprises stainless steel, a polymer, or a combination thereof to provide atraumatic transition for gum line cannulation.

3. The bioactive glass delivery system of claim 1, wherein the cannula includes an oval or rectangular cross-sectional shape.

4. The bioactive glass delivery system of claim 1, wherein the bioactive glass material comprises fiber, beads, or combinations thereof.

5. The bioactive glass delivery system of claim 1, wherein the trace elements are present at concentrations ranging from about 0.01% to about 20% by weight.

6. The bioactive glass delivery system of claim 1, wherein the obturator is configured to compact the bioactive glass material into periodontal pockets after delivery.

7. A method of stabilizing loose teeth, comprising:
accessing a periodontal void adjacent to a loose tooth;
delivering a bioactive glass composition into the periodontal void using a delivery system, wherein the bioactive glass composition comprises boron oxide and calcium oxide as primary network formers; and
allowing the bioactive glass composition to provide mechanical stabilization while releasing bioactive ions for bone regeneration.

8. The method of claim 7, wherein the bioactive glass composition is enhanced with one or more trace elements selected from copper oxide, zinc oxide, strontium oxide, iron oxide, potassium oxide, magnesium oxide, sodium oxide, and phosphorus pentoxide at concentrations of about 0.01% to about 20% by weight.

9. The method of claim 7, wherein the bioactive glass composition is configured to dissolve in interstitial fluids to create an alkaline environment.

10. The method of claim 7, wherein delivering the bioactive glass composition includes manipulating an obturator of the delivery system to compact the bioactive glass composition.

11. The method of claim 7, wherein the bioactive glass composition includes Type I collagen at concentrations of about 0.1% to about 30% by weight.

12. The method of claim 7, wherein accessing the periodontal void comprises scaling and root planing to prepare a periodontal pocket and remove bacterial deposits.

13. A phase-invertible gel composition for periodontal therapy, comprising:
bioactive glass particles comprising boron oxide and calcium oxide as primary network formers; and
a phase-invertible carrier in water at a concentration of about 30% by weight, wherein the phase-invertible gel composition is configured to transition from liquid to gel at body temperature.

14. The phase-invertible gel composition of claim 13, wherein the bioactive glass particles may be enhanced with trace elements at concentrations ranging from about 0.01% to about 20% by weight, wherein the trace elements may be selected from copper oxide, zinc oxide, strontium oxide, iron oxide, potassium oxide, magnesium oxide, sodium oxide, and phosphorus pentoxide.

15. The phase-invertible gel composition of claim 13, wherein the bioactive glass particles are added to the phase-invertible carrier at concentrations of about 10% to about 70% by weight.

16. The phase-invertible gel composition of claim 13, wherein the composition is prepared by compounding the phase invertible carrier at temperatures below 40° F. to form a viscous liquid with another syringe containing bioactive glass particles at temperatures below 40° F.

17. The phase invertible gel composition of claim 16, wherein the composition is configured to be injected through tissue adjacent to areas of bone loss and/or periodontal voids.

18. The phase-invertible gel composition of claim 13, wherein the phase inverted composition is configured to retain the bioactive glass particles at treatment sites.

19. The phase-invertible gel composition of claim 13 further comprising Type I collagen at concentrations of about 0.1% to about 5% by weight.

20. A method of creating antimicrobial conditions in periodontal pockets, comprising:
delivering the phase-invertible gel composition of claim 13 into a periodontal pocket;
allowing the phase-invertible carrier to dissolve while maintaining the bioactive glass particles in the periodontal pocket to facilitate an alkaline environment; and
inhibiting periodontal pathogens via the alkaline environment.
